(19) United States
(12) Patent Application Publication
(10) Pub. No.: US 2023/0377140 A1
(43) Pub. Date: Nov. 23, 2023

(54) Systems and Methods for Image-Based Disease Characterization

(71) Applicant: 4D Path Inc., Newton, MA (US)

(72) Inventors: Satabhisa Mukhopadhyay, Brookline, MA (US); Tathagata Dasgupta, Brookline, MA (US)

(21) Appl. No.: 18/104,695

(22) Filed: Feb. 1, 2023

Related U.S. Application Data
(60) Provisional application No. 63/410,588, filed on Sep. 27, 2022.
(60) Provisional application No. 63/305,540, filed on Feb. 1, 2022.

(51) Int. Cl. G06T 7/00; G06T 7/11

(52) U.S. Cl. CPC G06T 7/0012; G06T 7/11; G06T 2207/30024; G06T 2207/10056

(57) ABSTRACT

Presented herein are systems and methods for automated cytological evaluation of an image of an in vitro biological sample. More specifically, in certain embodiments, the present disclosure encompasses systems, methods, and apparatus for diagnostic evaluation of a two- dimensional image of a stained biological sample using cell cycle deformation functions.

### CROSS-REFERENCE TO RELATED APPLICATIONS

This application claims the benefit of the filing date of U.S. Provisional Patent Application Ser. No. 63/305,540 and U.S. Provisional Patent Application Ser. No. 63/410,588, which are both incorporated herein by reference in their entirety. Further, U.S. application Ser. No. 15/965,664 is also incorporated herein by reference in its entirety.

### TECHNICAL FIELD

This specification relates to automated cytological evaluation of an image of a biological sample.

### BACKGROUND

Images of biological samples can characterize various phases of the cell cycle, e.g., the M, G0, G1, S, and G2 phases. To divide, a cell must complete several tasks: it must grow, copy its genetic material (DNA), and physically split into two daughter cells. Cells perform these tasks in an organized, predictable series of steps that make up the cell cycle. The cell cycle is a cycle, rather than a linear pathway, because at the end of each go-round, the two daughter cells can start the exact same process over again from the beginning.

In general, cellular division (sometimes referred to as “cell cycling”) occurs in two distinct phases: interphase and mitosis (sometimes referred to as “M phase,” the “mitotic phase,” or simply “M”). Interphase consists of a G1 phase, an S phase, and a G2 phase. In G1 phase (sometimes referred to as the “first gap phase”), a cell is active and growing, and cells are receptive to signals to begin DNA synthesis. In S phase, a cell synthesizes a complete copy of the DNA in its nucleus and duplicates a microtubule-organizing structure called the centrosome. The centrosomes help separate DNA during M phase. In G2 phase (sometimes referred to as the “second gap phase”), a cell actively prepares for M phase and contains twice the normal amount of DNA in its nucleus.

### SUMMARY

In one aspect, described herein is a computer-implemented method comprising: receiving, by a processor of a computing device, a digital image of a biological sample, the digital image comprising an area segmented into a plurality of subsections; calculating, by the processor, a diagnostic score for a first subsection of the plurality of subsections, the diagnostic score comprising a plurality of values (e.g., vector values) collectively associated with a nuclear heat and a nuclear area of one or more cells imaged in the first subsection of the biological sample; executing, by the processor, a cell cycle deformation function to generate a corresponding diagnostic index for the first subsection, wherein the cell cycle deformation function identifies one or more cell cycle deformations based on a subset of the plurality of values of the diagnostic score for the first subsection; mapping, by the processor, the diagnostic index for the first subsection to a reference scale to determine whether the diagnostic index for the first subsection exceeds a threshold value on the cell cycle deformation reference scale; and determining, by the processor, a presence of one or more cell cycle deformations associated with the first subsection based on the diagnostic index exceeding the threshold value.

In certain embodiments, the method further comprises graphically denoting (e.g., highlighting), in the area of the digital image, the first subsection based on the diagnostic index exceeding the threshold value. In certain embodiments, the graphically denoting comprises generating a two-dimensional shape (e.g., a polygon) to be rendered on the digital image, the two-dimensional shape comprising a boundary (e.g., a continuous boundary) surrounding the first subsection based on the diagnostic index exceeding the threshold value.

In certain embodiments, the method further comprises generating one or more pathology annotations associated with the boundary (e.g., annotating the boundary for subsequent pathologist review). In certain embodiments, the boundary further surrounds one or more additional subsections of the plurality of subsections based on their respective diagnostic indices exceeding the threshold value. In certain embodiments, the boundary excludes one or more subsections whose corresponding diagnostic indices fail to exceed the threshold value. In certain embodiments, the method further comprises generating a heat map within the two-dimensional shape, wherein the heat map is illustrative of a degree or degrees to which the diagnostic index of the corresponding first subsection and the respective diagnostic indices of the corresponding one or more additional subsections exceed the threshold value. In certain embodiments, the two-dimensional shape comprises a convex hull polygon.

In certain embodiments, the executing comprises mapping the subset of the plurality of values of the diagnostic score to corresponding variables in the cell cycle deformation function.

In certain embodiments, an identified degree of the presence of cell cycle deformation in the first subsection is proportional to a degree to which the diagnostic index exceeds the threshold value.

In certain embodiments, the cell cycle deformation function calculates cell cycle S-phase deregulation to identify regions of high grade cancer [e.g., by quantifying Shape (<C>) junction curvature variance] (e.g., to identify a region containing mitotic figures).

In certain embodiments, the cell cycle deformation function calculates active cell cycle to identify regions of high percentage of Ki67 expression [e.g., by quantifying (skew in C_L-initial)×(skew in C_L-max)] (e.g., to identify a region containing mitotic figures).

In certain embodiments, the cell cycle deformation function calculates skew in nuclear size and chromosomal instability to identify regions of high DNA ploidy [e.g., by quantifying percentage of tissue area with high skew in (C_L)max].

In certain embodiments, the cell cycle deformation function calculates structural distortion in stroma and cell cycle arrest to identify regions of high stromal TILs [e.g., by quantifying shape (<C>) deviation].

In certain embodiments, the cell cycle deformation function calculates cell cycle G1/S deregulation and arrest signature to identify HER2 positive/amplified regions [e.g., by quantifying shape (<C_L>) divergence].

In certain embodiments, the cell cycle deformation function calculates cell cycle G1/S deregulation and arrest signature to identify HR positive/overexpressed regions [e.g., by quantifying shape (<C>) divergence].

In certain embodiments, the cell cycle deformation function calculates cell cycle G1entry and G0 arrest signature to identify regions harboring high Quiescent Population Load (QPL) [e.g., by quantifying (<C>max−<C>min)×(skew in <C>max)].

In certain embodiments, the cell cycle deformation function calculates chromosomal instability to identify BRCA positive and HRD harboring regions [e.g., by quantifying jump in shape (<C>) bound].

In certain embodiments, the cell cycle deformation function calculates cell cycle arrest and chromosomal instability to identify regions harboring MMR deficiency [e.g., by quantifying shape (<C>) section slope fluctuation].

In certain embodiments, the graphically denoting comprises rendering an overlay on the digital image, said overlay graphically identifying one or more regions of the digital image corresponding to cell abnormality.

In certain embodiments, the method comprises automatically identifying, by the processor, one or more edges of the biological sample depicted in the digital image and confining the two-dimensional shape to a region within the one or more edges of the biological sample.

In certain embodiments, the image of the biological sample is a stained tissue image (e.g., a two-dimensional image, e.g., a hematoxylin and eosin stained formalin fixed paraffin embedded tissue image [an H&E stained FFPE image]).

In certain embodiments, the method comprises executing, by the processor, each of one or more cell cycle deformation functions to generate a corresponding diagnostic index for each of the plurality of segmented subsections of the digital image, wherein the one or more cell cycle deformation functions (collectively) determine one or more diagnostic entities selected from the group consisting of: cancer histological grade, mitotic figures, Ki67 status, DNA ploidy, stromal TILs status, HER2 status, HR (combined ER, PR) status, Quiescent Population Load (QPL), BRCA mutation or HRD status, MMR deficiency status, or any combination of the foregoing.

In certain embodiments, the method comprises executing, by the processor, each of one or more cell cycle deformation functions to generate a corresponding diagnostic index for each of the plurality of segmented subsections of the digital image, wherein the one or more cell cycle deformation functions (collectively) calculate one or more surrogate biological signatures selected from the group consisting of: deregulation of cell cycle DNA synthesis phase (S-phase), cell cycle active phase length, M-phase activity, skew in nuclear size and chromosomal instability, structural distortion in stroma and overall measure of cell cycle arrest, cell cycle G1/S deregulation and arrest signature, cell cycle G1 entry and G0 arrest signature, chromosomal instability, cell cycle arrest, and or combination of the foregoing.

In another aspect, described herein is a method comprising: receiving, by a processor of a computing device, a digital image of a biological sample, and automatically evaluating, by the processor, a measure of cell cycle S-phase deregulation for each of at least one subsection of the digital image to identify regions of high grade cancer (e.g., wherein the digital image is segmented into a plurality of subsections corresponding to different regions of the imaged biological sample).

In certain embodiments, the automatically evaluating comprises computing a Shape (<C>) junction curvature variance for each of the at least one subsection.

In certain embodiments, the method comprises identifying one or more regions of the digital image containing mitotic figures using the measure of cell cycle S-phase deregulation.

In another aspect, described herein is a method comprising: receiving, by a processor of a computing device, a digital image of a biological sample, and automatically evaluating, by the processor, a measure of active cell cycle of each of at least one subsection of the digital image to identify regions of high percentage of Ki67 expression (e.g., wherein the digital image is segmented into a plurality of subsections corresponding to different regions of the imaged biological sample).

In certain embodiments, the automatically evaluating comprises computing (skew in C_L-initial)×(skew in C_L-max) for each of the at least one subsection.

In certain embodiments, the method comprises identifying one or more regions of the digital image containing mitotic figures using the measure of active cell cycle.

In another aspect, described herein is a method comprising: receiving, by a processor of a computing device, a digital image of a biological sample, and automatically evaluating, by the processor, a measure of skew in nuclear size and chromosomal instability of each of at least one subsection of the digital image to identify regions of high DNA ploidy (e.g., wherein the digital image is segmented into a plurality of subsections corresponding to different regions of the imaged biological sample).

In certain embodiments, the automatically evaluating comprises computing a percentage of tissue area with high skew in (C_L)max for each of the at least one subsection.

In another aspect, described herein is a method comprising: receiving, by a processor of a computing device, a digital image of a biological sample, and automatically evaluating, by the processor, a measure of structural distortion in stroma and cell cycle arrest of each of at least one subsection of the digital image to identify regions of high stromal TILs (e.g., wherein the digital image is segmented into a plurality of subsections corresponding to different regions of the imaged biological sample).

In certain embodiments, the automatically evaluating comprises computing shape (<C>) deviation for each of the at least one subsection.

In another aspect, described herein is a method comprising: receiving, by a processor of a computing device, a digital image of a biological sample, and automatically evaluating, by the processor, a measure of cell cycle G1/S deregulation and arrest signature of each of at least one subsection of the digital image to identify HER2 positive/amplified regions (e.g., wherein the digital image is segmented into a plurality of subsections corresponding to different regions of the imaged biological sample).

In certain embodiments, the automatically evaluating comprises computing shape (<C_L>) divergence for each of the at least one subsection.

In another aspect, described herein is a method comprising: receiving, by a processor of a computing device, a digital image of a biological sample, and automatically evaluating, by the processor, a measure of cell cycle G1/S deregulation and arrest signature of each of at least one subsection of the digital image to identify HR positive/overexpressed regions (e.g., wherein the digital image is segmented into a plurality of subsections corresponding to different regions of the imaged biological sample).

In certain embodiments, the automatically evaluating comprises computing shape (<C>) divergence for each of the at least one subsection.

In another aspect, described herein is a method comprising: receiving, by a processor of a computing device, a digital image of a biological sample, and automatically evaluating, by the processor, a measure of cell cycle G1 entry and G0 arrest signature of each of at least one subsection of the digital image to identify regions harboring high quiescent population load (QPL) (e.g., wherein the digital image is segmented into a plurality of subsections corresponding to different regions of the imaged biological sample). In certain embodiments, the automatically evaluating comprises computing (<C>max−<C>min)×(skew in <C>max) for each of the at least one subsection.

In another aspect, described herein is a method comprising: receiving, by a processor of a computing device, a digital image of a biological sample, and automatically evaluating, by the processor, a measure of chromosomal instability of each of at least one subsection of the digital image to identify BRCA positive and HRD harboring regions (e.g., wherein the digital image is segmented into a plurality of subsections corresponding to different regions of the imaged biological sample).

In certain embodiments, the automatically evaluating comprises computing a jump in shape (<C>) bound for each of the at least one subsection.

In another aspect, described herein is a method comprising: receiving, by a processor of a computing device, a digital image of a biological sample, and automatically evaluating, by the processor, a measure of cell cycle arrest and chromosomal instability of each of at least one subsection of the digital image to identify regions harboring MMR deficiency (e.g., wherein the digital image is segmented into a plurality of subsections corresponding to different regions of the imaged biological sample).

In certain embodiments, the automatically evaluating comprises computing a shape (<C>) section slope fluctuation for each of the at least one subsection.

In another aspect, described herein is a system comprising: a processor of a computing device; and a memory having instructions stored thereon, wherein the instructions, when executed by the processor, cause the processor to perform one or more steps of the methods described herein.

In another aspect, described herein is a computer-implemented method comprising: determining, by a processor of a computing device, a presence of one or more cell cycle deformations associated with a first subsection of a digital image of a biological sample, the digital image comprising an area segmented into a plurality of subsections, wherein the determining is based on a diagnostic index exceeding a threshold value, wherein a diagnostic score for the first subsection comprises a plurality of values (e.g., vector values) collectively associated with a nuclear heat and a nuclear area of one or more cells imaged in the first subsection of the biological sample, and wherein the diagnostic index for the first subsection is determined using a cell cycle deformation function that identifies one or more cell cycle deformations based on a subset of a plurality of values of the diagnostic score for the first subsection.

In certain embodiments, the cell cycle deformation function calculates a surrogate biological signature selected from the group consisting of: deregulation of cell cycle DNA synthesis phase (S-phase), cell cycle active phase length, M-phase activity, skew in nuclear size and chromosomal instability, structural distortion in stroma and overall measure of cell cycle arrest, cell cycle G1/S deregulation and arrest signature, cell cycle G1 entry and G0 arrest signature, chromosomal instability, cell cycle arrest, or any combination of the foregoing.

In certain embodiments, the cell cycle deformation function determines a diagnostic entity selected from the group consisting of: cancer histological grade, mitotic figures, Ki67 status, DNA ploidy, stromal TILs status, HER2 status, HR (combined ER, PR) status, quiescent population load (QPL), BRCA mutation or HRD status, MMR deficiency status, or any combination of the foregoing.

In certain embodiments, the method further comprises automatically identifying and graphically rendering, by the processor, a two-dimensional shape (e.g., a polygon) comprising a boundary (e.g., a continuous boundary) surrounding the first subsection and one or more additional subsections of the digital image based on the diagnostic index of each of the first subsection and each of the additional subsections exceeding the threshold value.

In certain embodiments, the cell cycle deformation function calculates cell cycle G1/S deregulation and cell cycle G1 phase entry deregulation to identify regions comprising one or more of BRAF and NRAS gene mutations.

According to another aspect, there is provided a method comprising: receiving, by a processor of a computing device, a digital image of a biological sample, and automatically evaluating, by the processor, a measure of cell cycle G1/S deregulation and cell cycle G1 phase entry deregulation of each of at least one subsection of the digital image to identify regions comprising one or more of BRAF and NRAS mutations.

In certain embodiments, automatically evaluating comprises computing shape (<C>max, <C>min, <C>) divergence for each of the at least one subsection.

In certain embodiments, the cell cycle deformation function calculates cell cycle G1/S deregulation to identify regions of dysplasia.

According to another aspect, there is provided a method comprising receiving, by a processor of a computing device, a digital image of a biological sample, and automatically evaluating, by the processor, a measure of cell cycle G1/S deregulation of each of at least one subsection of the digital image to identify regions of dysplasia.

In certain embodiments, automatically evaluating comprises computing shape (<C_L>, <C>) divergence for each of the at least one subsection.

In certain embodiments, the cell cycle deformation function calculates cell cycle G2/M deregulation to identify degree of dysplasia in regions.

According to another aspect, there is provided a method comprising: receiving, by a processor of a computing device, a digital image of a biological sample, and automatically evaluating, by the processor, a measure of cell cycle G2/M deregulation of each of at least one subsection of the digital image to identify degree of dysplasia in regions of the digital image.

In certain embodiments, automatically evaluating comprises computing shape (<C_L>, <C>max) divergence & (<C>max−<C>min) shift for each of the at least one subsection.

The details of one or more embodiments of the subject matter of this specification are set forth in the accompanying drawings and the description below. Other features, aspects, and advantages of the subject matter will become apparent from the description, the drawings, and the claims.

### DEFINITIONS

For the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers, or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “cancer” refers to a disease, disorder, or condition in which cells exhibit relatively abnormal, uncontrolled, or autonomous growth, so that they display an abnormally elevated proliferation rate or aberrant growth phenotype characterized by a significant loss of control of cell proliferation. In some embodiments, a cancer is characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma,gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T-cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva. For example, tissue images shown in the figures include endometrial cancer (FIG. 15) and breast cancer (all others).

As used herein the term “detector” includes any detector of electromagnetic radiation including, but not limited to, CCD camera, photomultiplier tubes, photodiodes, and avalanche photodiodes.

Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration or manipulation of data or information, for example utilizing a computer or other processing unit adapted to perform a relevant analysis. In some embodiments, determining involves receiving relevant information or materials from a source. In some embodiments, determining involves comparing one or more features of a sample or entity to a comparable reference. Similarly, a step of “calculating”, “executing”, or “mapping” can utilize or be accomplished in a manner as discussed above with respect to a step of “determining”.

As used herein, “diagnosis” refers to providing any type of diagnostic information, including, but not limited to, whether a subject is likely to have or develop a disease, disorder or condition, state, staging or characteristic of a disease, disorder, or condition as manifested in the subject, information related to the nature or classification of a tumor, information related to prognosis or information useful in selecting an appropriate treatment. Selection of treatment may include the choice of a particular therapeutic agent or other treatment modality such as surgery, radiation, etc., a choice about whether to withhold or deliver therapy, a choice relating to dosing regimen (e.g., frequency or level of one or more doses of a particular therapeutic agent or combination of therapeutic agents), etc.

As used herein, the term “electromagnetic radiation” or “radiation” is understood to mean self-propagating waves in space of electric and magnetic components that oscillate at right angles to each other and to the direction of propagation, and which are in phase with each other. Electromagnetic radiation includes: radio waves, microwaves, red, infrared, and near-infrared light, visible light, ultraviolet light, X-rays and gamma rays.

As used herein, the term “genotype” refers to the diploid combination of alleles at a given genetic locus, or set of related loci, in a given cell or organism. A homozygous subject carries two copies of the same allele and a heterozygous subject carries two distinct alleles. In the simplest case of a locus with two alleles “A” and “a”, three genotypes can be formed: A/A, A/a, and a/a.

As used herein, an “image”—for example, a two-dimensional or three-dimensional image of an in vitro biological sample such as tissue—includes any visual representation, such as a photo, a video frame, streaming video, as well as any electronic, digital or mathematical analogue of a photo, video frame, or streaming video. Any apparatus described herein, in certain embodiments, includes a display for displaying an image or any other result produced by the processor. Any method described herein, in certain embodiments, includes a step of displaying an image or any other result produced via the method.

The term “in vitro” as used herein refers to events that occur in an artificial environment, e.g., in a test tube or reaction vessel, in cell culture, etc., rather than within a multi-cellular organism.

The term “in vivo” as used herein refers to events that occur within a multi-cellular organism, such as a human and a non-human animal. In the context of cell-based systems, the term may be used to refer to events that occur within a living cell (as opposed to, for example, in vitro systems).

As used herein, the term “map” is understood to mean a visual display, or any data representation that may be interpreted for visual display, which contains spatially-correlated information. For example, a three-dimensional map of a given volume may include a dataset of values of a given quantity that varies in three spatial dimensions throughout the volume. A three-dimensional map may be displayed in two-dimensions (e.g., on a two-dimensional screen, or on a two-dimensional printout).

As used herein, the term “phenotype” refers to a trait, or to a class or set of traits displayed by a cell or organism. In some embodiments, a particular phenotype may correlate with a particular allele or genotype. In some embodiments, a phenotype may be discrete; in some embodiments, a phenotype may be continuous.

As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on or comparison to a particular possible reference or control.

As used herein, the term “sample” typically refers to an aliquot of material obtained or derived from a source of interest, as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, the biological tissue is an in vitro sample prepared for two-dimensional or three- dimensional imaging or other cytology tests. For example, the sample may be stained to better reveal structures of the cells of the sample such as the nucleus, the cytoplasm, and cellular granules. Various staining methods or other sample processing such as fixation, dehydration, clearing, and slide mounting may be used. For example, the sample may be a hematoxylin and eosin stained (H&E) stained sample, or the sample may comprise formalin fixed paraffin embedded (FFPE) tissue. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, or combinations or component(s) thereof. In some embodiments, a biological fluid may be or comprise an intracellular fluid, an extracellular fluid, an intravascular fluid (blood plasma), an interstitial fluid, a lymphatic fluid, or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation or purification of certain components, etc.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion or proceed to completeness or achieve or avoid an absolute result. The term “substantially” is therefore used herein to capture the potential lack of completeness inherent in many biological and chemical phenomena.

### BRIEF DESCRIPTION OF DRAWINGS

The foregoing and other objects, aspects, features, and advantages of the present disclosure will become more apparent and better understood by referring to the following description taken in conjunction with the accompanying drawings, in which:

FIG. 1 is a schematic diagram of a diagnostic procedure involving the digital analysis of a two-dimensional image of a stained biological sample, according to an illustrative embodiment.

FIG. 2 is a schematic diagram of a cell cycle, presented for contextual purposes.

FIG. 3 is a block flow diagram of a diagnostic procedure for cytological analysis of an image of an in vitro biological sample, according to an illustrative embodiment.

FIG. 4A-4F are images of a biological sample and corresponding plots illustrating quantification of cell cycle deformation to identify regions carrying morphometric features of clinical interest, such as Grade, according to an illustrative embodiment.

FIG. 5A-5D are images of a biological sample with computed, rendered graphical overlays demonstrating user interfaces such as diagnostic boundary back-projection, pathology annotation, and graphical heat maps, according to an illustrative embodiment.

FIG. 6A-6E are images of a biological sample and corresponding plots illustrating determination of a cell cycle deformation function (Function 1) for calculating cell cycle S-phase deregulation to identify regions of high Grade cancer, according to an illustrative embodiment.

FIG. 7A-7E are images of a biological sample and corresponding plots illustrating determination of a cell cycle deformation function (Function 2) for calculating active cell cycle to identify regions of high percentage of Ki67 expression, according to an illustrative embodiment.

FIG. 8A-8C are images of a biological sample and corresponding overlays illustrating use of cell cycle deformation Functions 1 and 2 above for identifying regions containing mitotic figures, according to an illustrative embodiment.

FIG. 9A-9E are images of a biological sample and corresponding plots illustrating determination of a cell cycle deformation function (Function 3) for calculating skew in nuclear size and chromosomal instability to identify regions of high DNA ploidy, according to an illustrative embodiment.

FIG. 10A-10E are images of a biological sample and corresponding plots illustrating determination of a cell cycle deformation function (Function 4) for calculating structural distortion in stroma and cell cycle arrest to identify regions of high stromal TILs, according to an illustrative embodiment.

FIG. 11A-11E are images of a biological sample and corresponding plots illustrating determination of a cell cycle deformation function (Function 5) for calculating cell cycle G1/S deregulation and arrest signature to identify HER2 positive/amplified regions, according to an illustrative embodiment.

FIG. 12A-12E are images of a biological sample and corresponding plots illustrating determination of a cell cycle deformation function (Function 6) for calculating cell cycle G1/S deregulation and arrest signature to identify HR positive/overexpressed regions, according to an illustrative embodiment.

FIG. 13A-13E are images of a biological sample and corresponding plots illustrating determination of a cell cycle deformation function (Function 7) for calculating cell cycle G1 entry and G0 arrest signature to identify regions harboring high quiescent population load (QPL), according to an illustrative embodiment.

FIG. 14A-14F are images of a biological sample and corresponding plots and heat map overlay illustrating determination of a cell cycle deformation function (Function 8) for calculating chromosomal instability to identify BRCA positive and HRD harboring regions, according to an illustrative embodiment.

FIG. 15A-15E are images of a biological sample and corresponding plots illustrating determination of a cell cycle deformation function (Function 9) for calculating cell cycle arrest and chromosomal instability to find regions harboring MMR deficiency, according to an illustrative embodiment.

FIG. 16 is a block flow diagram of a method for determining a presence of one or more cell cycle deformations associated with each of one or more subsections of a digital image of a biological sample, according to an illustrative embodiment.

FIG. 17 is a block flow diagram of a method for automatically evaluating a measure of cell cycle S-phase deregulation for each of at least one subsection of a digital image of a biological sample, according to an illustrative embodiment.

FIG. 18 is a block flow diagram of a method for automatically evaluating a measure of active cell cycle for each of at least one subsection of a digital image of a biological sample, according to an illustrative embodiment.

FIG. 19 is a block flow diagram of a method for automatically evaluating a measure of skew in nuclear size and chromosomal instability for each of at least one subsection of a digital image of a biological sample, according to an illustrative embodiment.

FIG. 20 is a block flow diagram of a method for automatically evaluating a measure of structural distortion in stroma and cell cycle arrest for each of at least one subsection of a digital image of a biological sample, according to an illustrative embodiment.

FIG. 21 is a block flow diagram of a method for automatically evaluating a measure of cell cycle G1/S deregulation and arrest signature for each of at least one subsection of a digital image of a biological sample to identify HER2 positive/amplified regions, according to an illustrative embodiment.

FIG. 22 is a block flow diagram of a method for automatically evaluating a measure of cell cycle G1/S deregulation and arrest signature for each of at least one subsection of a digital image of a biological sample to identify HR positive/overexpressed regions, according to an illustrative embodiment.

FIG. 23 is a block flow diagram of a method for automatically evaluating a measure of cell cycle G1 entry and G0 arrest signature for each of at least one subsection of a digital image of a biological sample, according to an illustrative embodiment.

FIG. 24 is a block flow diagram of a method for automatically evaluating a measure of chromosomal instability for each of at least one subsection of a digital image of a biological sample, according to an illustrative embodiment.

FIG. 25 is a block flow diagram of a method for automatically evaluating a measure of cell cycle arrest and chromosomal instability for each of at least one subsection of a digital image of a biological sample, according to an illustrative embodiment.

FIG. 26 is a block flow diagram of a method for determining a presence of one or more cell cycle deformations associated with each of at least one subsection of a digital image of a biological sample, according to an illustrative embodiment.

FIG. 27 is a schematic diagram of a system and associated method for obtaining and analyzing a digital image of a biological sample, according to an illustrative embodiment.

FIG. 28A-28E are images of a biological sample and corresponding plots illustrating determination of a cell cycle deformation function (Function 10) for calculating cell cycle G1 entry and G1/S deformation to find regions harboring BRAF/NRAS mutations, according to an illustrative embodiment.

FIG. 29 shows a heat map of BRAF/NRAS mutation status in malignant melanoma.

FIG. 30 is a block flow diagram of a method for automatically evaluating a measure of cell cycle G1/S deregulation and cell cycle G1 phase entry deregulation for each of at least one subsection of a digital image of a biological sample, according to an illustrative embodiment.

FIG. 31A-31F are images of a biological sample and corresponding plots illustrating determination of cell cycle deformation functions (Function 11 and Function 12) for calculating cell cycle G1/S deregulation signature and cell cycle G2/M deregulation signature to identify dysplasia and degree of dysplasia in Barrett's Esophagus.

FIG. 32 is a block flow diagram of a method for automatically evaluating a measure of cell cycle G1/S deregulation of each of at least one subsection of a digital image of a biological sample to identify regions of dysplasia, according to an illustrative embodiment.

FIG. 33 is a block flow diagram of a method for automatically evaluating a measure of cell cycle G2/M deregulation of each of at least one subsection of the digital image to identify degree of dysplasia in regions of the digital image, according to an illustrative embodiment.

### DETAILED DESCRIPTION

Throughout the description, where compositions are described as having, including, or comprising specific components, or where methods are described as having, including, or comprising specific steps, it is contemplated that, additionally, there are compositions of the present specification that consist essentially of, or consist of, the recited components, and that there are methods according to the present specification that consist essentially of, or consist of, the recited processing steps.

The order of steps or order for performing certain action is immaterial so long as the result of the associated process or its overall functionality remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

All references cited herein, including patent applications and publications, are incorporated by reference in their entirety.

Presented herein are systems and methods for automated cytological evaluation of an image of an in vitro biological sample. More specifically, in certain embodiments, the present disclosure encompasses systems, methods, and apparatus for diagnostic evaluation of a two-dimensional image of a stained biological sample using cell cycle deformation functions.

FIG. 2 is a schematic diagram of a cell cycle, presented for contextual purposes. The diagram illustrates various phases of the cell cycle—the M, G0, G1, S, and G2 phases—explained herein in more detail. To divide, a cell must complete several tasks: it must grow, copy its genetic material (DNA), and physically split into two daughter cells. Cells perform these tasks in an organized, predictable series of steps that make up the cell cycle. The cell cycle is a cycle, rather than a linear pathway, because at the end of each go-round, the two daughter cells can start the exact same process over again from the beginning. In general, cellular division (sometimes referred to as “cell cycling”) occurs in two distinct phases: interphase and mitosis (sometimes referred to as “M phase,” the “mitotic phase,” or simply “M”). Interphase consists of a G1 phase, an S phase, and a G2 phase. In G1 phase (sometimes referred to as the “first gap phase”), a cell is active and growing, and cells are receptive to signals to begins DNA synthesis. In S phase, a cell synthesizes a complete copy of the DNA in its nucleus and duplicates a microtubule-organizing structure called the centrosome. The centrosomes help separate DNA during M phase. In G2 phase (sometimes referred to as the “second gap phase”), a cell actively prepares for M phase and contains twice the normal amount of DNA in its nucleus.

M phase occurs in a four-step process: (1) prophase, (2) metaphase, (3) anaphase, and (4) telophase. In prophase (the first stage of cell division) chromosomes become visible as paired chromatids and the nuclear envelope disappears. The first prophase of meiosis includes the reduction division. In metaphase (the second stage of cell division) the chromosomes become attached to the spindle fibers. In anaphase, (the third stage of cell division), the chromosomes move away from one another to opposite poles of the spindle. Finally, in telophase (the final phase of cell division), the chromatids or chromosomes move to opposite ends of the cell such that two nuclei are formed, which enables the eventual separation of the parent cell to form two daughter cells.

After the formation of the two daughter cells, some types of cells divide rapidly and, in such cases, the daughter cells may immediately undergo another round of cell division. For instance, many cell types in an early embryo divide rapidly, and so do cells in a tumor. Other types of cells divide slowly or not at all. These cells may exit the G1 phase and enter a resting state called G0. A cell in G0 has exited mitosis and is quiescent. That is, in G0, a cell may not be actively preparing to divide, and may instead be simply performing its function. For instance, a cell in G0 might conduct signals as a neuron or store carbohydrates as a liver cell. For some cells, G0 is a permanent state while others may re-start division if they receive the right signals from their environment.

The subject specification includes techniques for performing diagnostic tests to calculate biological signatures of various molecular profiles of a biological sample, such as detecting cell abnormalities (or the absence thereof). In some examples, the present disclosure describes techniques for using measured cell characteristics (e.g., nuclear contrast features or nuclear area features and the techniques for calculating and quantifying same, such as those described in U.S. application Ser. No. 15/965,664) to characterize, or to determine the presence, absence, or degree of: (1) cell cycle deregulations, such as DNA synthesis phase (S-phase) deregulation, to determine the extent of cancer (histological) grade and thereby identify regions of high cancer grade (grade 3 or grade 2 cancer, as appropriate for the clinical presentation), as well as regions with mitotic figures, (2) active cell cycles durations to determine the extent of percentage Ki67 expression by the cancerous tissue and thereby identify regions of high percentage Ki67 expression, as well as regions with mitotic figures, (3) skew in nuclear size and chromosomal instability to determine the extent of DNA Ploidy and thereby identify regions of high DNA Ploidy, (4) structural distortion in stroma and cell cycle arrest to determine the extent of the stromal Tumor Infiltrating Lymphocytes (TILs) and thereby identify regions of high stromal TILs, (5) certain cell cycle growth one (G1) phase to the S-phase transition (G1/S transition) deregulation and cell cycle arrest signatures to determine the extent of HER2 amplification or expression status and thereby identify HER2 positive (amplified or overexpressed) regions, (6) certain cell cycle G1/S transition deregulation and cell cycle arrest signatures to determine the extent of Hormone Receptor (HR) expression status (which is a combined Estrogen Receptor (ER) and Progesterone Receptor (PR) expression status) and thereby identify HR positive (ER or PR overexpressed) regions), (7) cell cycle G1-phase entry and G0-phase arrest signature to determine the extent of “quiescent cell population load” (QPL) and thereby identify regions harboring high QPL, (8) chromosomal instability to determine the extent of BRCA mutational status and Homologous Recombination Deficiency (HRD) status, (also referred to as “BRCA-ness”), and thereby identify regions harboring BRCA mutation and HRD, and (9) cell cycle arrest and chromosomal instability to determine the extent of mismatch repair (MMR) deficiency and identify MMR deficient regions. In each and all of the above cases the regions of interest are identified using one or multiple thresholds.

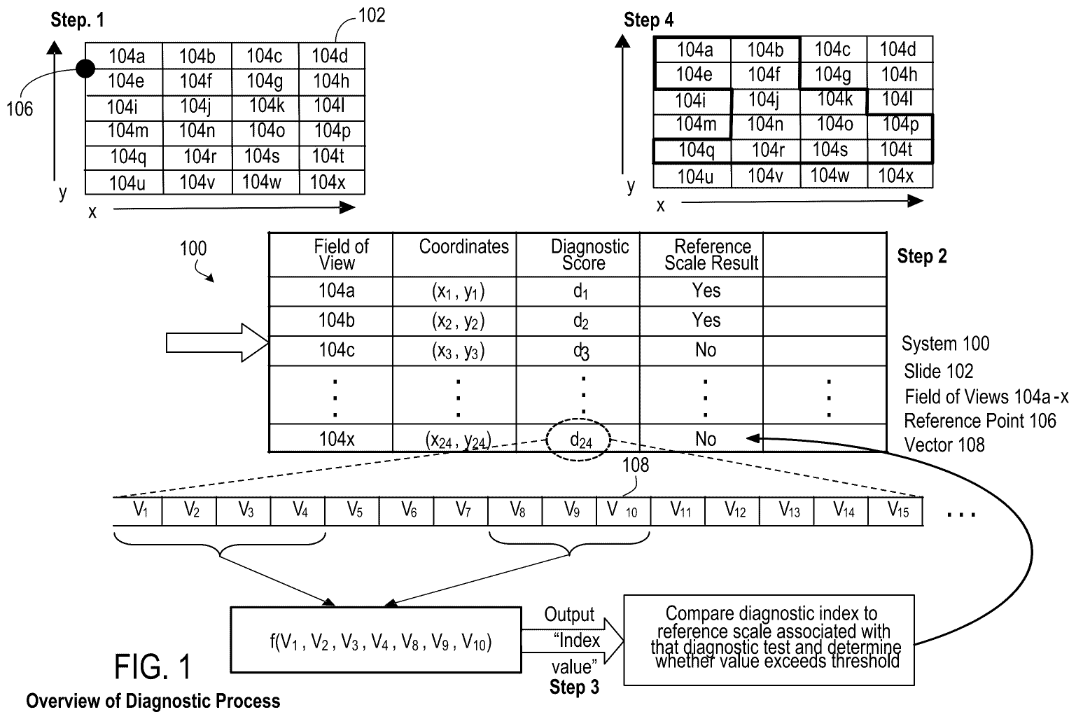

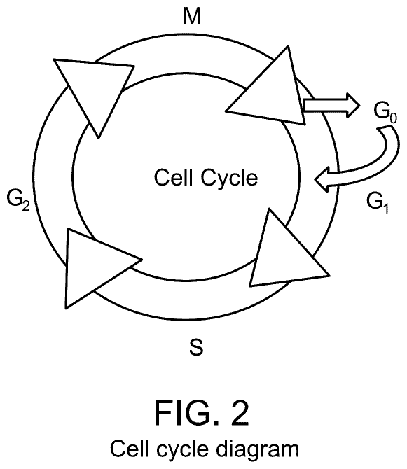

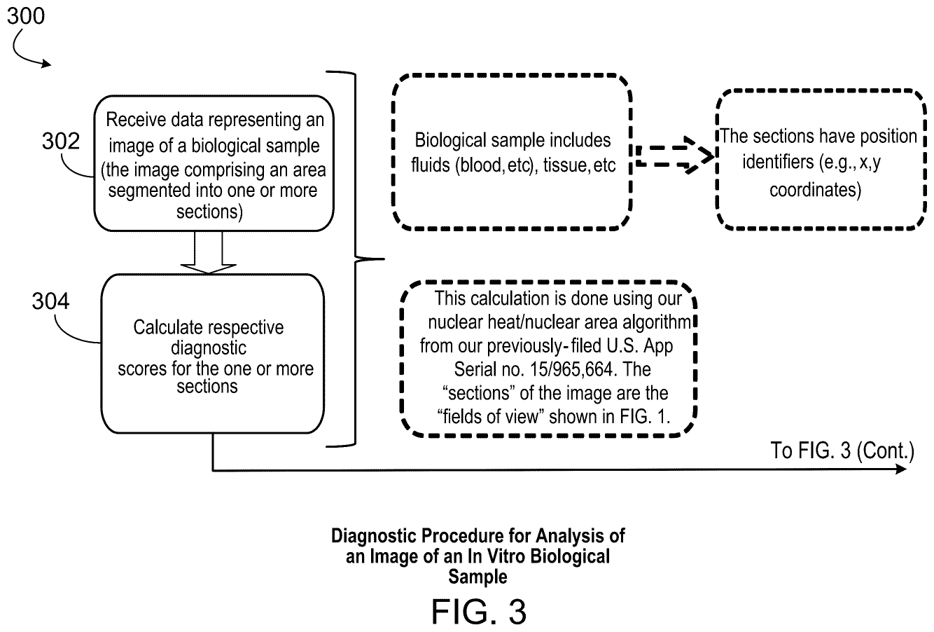

Step 1 of the diagnostic procedure 100 illustrated in FIG. 1 is receiving a digital image of a segmented biological sample (or segmenting the image). Segments (also referenced herein as “subsections” or “fields of view”, 104a-104x) of the slide image 102 that correspond to an area of the imaged biological sample are identified by an (x, y) coordinate reference point 106—in this example, the coordinate of the lower left corner of each field/subsection.

Next, in Step 2 of the procedure 100, a diagnostic score is calculated for each field of view using nuclear heat/nuclear area algorithms, examples of which are described throughout this specification. The diagnostic score for each field of view is a vector of values each representing a different measurement or characteristic. In the example, fifteen values are shown for example diagnostic score d24.

In certain embodiments, the thermal and thermodynamic diagnostic parameters (e.g., that are included in the diagnostic scores) are computed from the ensemble (e.g., ensemble average) dynamics of the total heat generated at different cellular compartments, their contrasts and respective area or other size parameters. In certain embodiments, the thermal and thermodynamic diagnostic parameters are computed as a function of all wavelengths (e.g., a function averaged over the entire range of naturally emitted IR wavelengths from a cell or cells thereof). An exemplary method described herein includes identifying one or more cells in the image (e.g., an emitted IR image, an H&E image) of a subject sample (e.g., tissue or fluid). Each of the cells in a field of view is segmented into a cellular area and a nuclear area. A nuclear area feature (α) is related to the ratio between a nuclear area and a nuclear volume projection. For example, the nuclear area feature (α) may be a function of a number of pixels within a nuclear area. A nuclear contrast feature (δh) is related to the hotness of the nuclear area with respect to the cellular area, the nuclear chromatin content, and/or the temperature of the nuclear area. For example, the nuclear contrast feature (δh) may be related to an average of pixel intensities within a nuclear area, which are higher than normal cytoplasmic intensity (e.g., an average of the pixel intensity within cytoplasmic area of normal healthy cells in a field of view). In certain embodiments, the nuclear contrast feature and/or the nuclear area feature is obtained at the wavelength corresponding to the maximal radiant power of the naturally emitted IR. The nuclear contrast feature may be an average of multiple nuclear contrast features obtained at multiple wavelengths of the naturally emitted IR. The nuclear area feature may be an averaged value of multiple nuclear area features obtained at multiple wavelengths of the naturally emitted IR.

In certain embodiments, an information surface value (S) for each cell is calculated from spatial probability densities (e.g., Ω(a), Ω(δh), Ω(δh×α), Ω(δh, α)) of the nuclear contrast feature (δh) and the nuclear area feature (α). For example, information surface value (S) may be

$$S = - log ( \frac{Ω ( \partial h \times a )}{Ω ( \partial h ) Ω ( a )} ) \qquad ( 1 )$$

In certain embodiments, the spatial probability densities of each cell is proportional to or a function of a fraction of cells. For example, the fraction of cells may be a ratio of a number of cells having a certain nuclear area or a range of nuclear area (e.g., greater than 5% of the median nuclear area) to the total number of cells in a field of view.

In certain embodiments, the cells in the subject sample image are divided into (e.g., characterized as) one or more subgroups depending on the information surface values (e.g., dimensionally reduced information surface values). The subgroups may correspond to different cell cycle stages and/or different intrinsic cell cycle time (L). In certain embodiments, the intrinsic cell cycle time (L) is related to chromatin content in a cell. For example, the intrinsic cell cycle time (L) is a function of the nuclear area feature and the nuclear contrast feature.

In certain embodiments, the thermal and thermodynamic diagnostic parameter includes a specificity index (SI). The specificity index may be related to an ensemble average of local specific heats among cells (<c_{L}>) in each of the subgroups. The subgroups may be determined by, for example, the information surface values, extremization of the information surface values, and/or one or more intrinsic cell time (L) of the information surface values. In certain embodiments, the specificity index is an integration of the ensemble average of the local specific heats over cell cycle stages and/or intrinsic cell cycle time, e.g.:

[symbol]

=η(ensemble average of local specific heat)dL (2)

In certain embodiments, the thermal and thermodynamic diagnostic parameter is a log thermal capacity (C). The log thermal capacity (C) may be related to a log of local specific heat among cells per unit area. In certain embodiments, the log thermal capacity (C) is an integration of the logarithm of the local specific heats among cells over cell cycle stages and/or intrinsic cell cycle time.

C=∫log(local specific heat among cells)dL (3)

In certain embodiments, a normality status of the subject tissue is determined by a diagnostic score (e.g., calculated from the thermal and thermodynamic decision parameters). In certain embodiments, the diagnostic score is mapped on a reference diagnostic scale. The reference diagnostic scale may include pre-assigned values for different types of cancers and their respective subtypes, benign inflammations, and various normal healthy conditions. In certain embodiments, normal healthy tissues have low positive score or negative score in the reference diagnostic scale. In certain embodiments, the diagnostic score (F) is a function of the thermal and thermodynamic diagnostic parameter.

F=g[<c_{L}>,<c_{L}>ₘₐₓ, [symbol] ,shape(<c_{L}>),<C>,shape(<C>)] (4)

where <c_{L}>ₘₐₓ is a maximum value of <c_{L}> in a given field of view, shape(<c_{L}>) is a shape feature of <c_{L}>, and shape (<C>) is a shape feature of <C>. In certain embodiments, the shape feature is avalue representing a shape of a curve of L vs <c_{L}> or L vs <C>. For example, when the curve is a piecewise function, the shape feature is a number of subsections, a value of L at junctions, a curvature at a junction of the subsections, relative <c_{L}> or <C> between consecutive subsections, or critical exponents capturing nature of singularities if any at any of such subsections, for example.

In the example shown in FIG. 1, the diagnostic score for each field of view is a vector 108 of values each representing a different measurement or characteristic. In this example, the vector has 15 values (V1-V15), but different embodiments may have a different number of values.

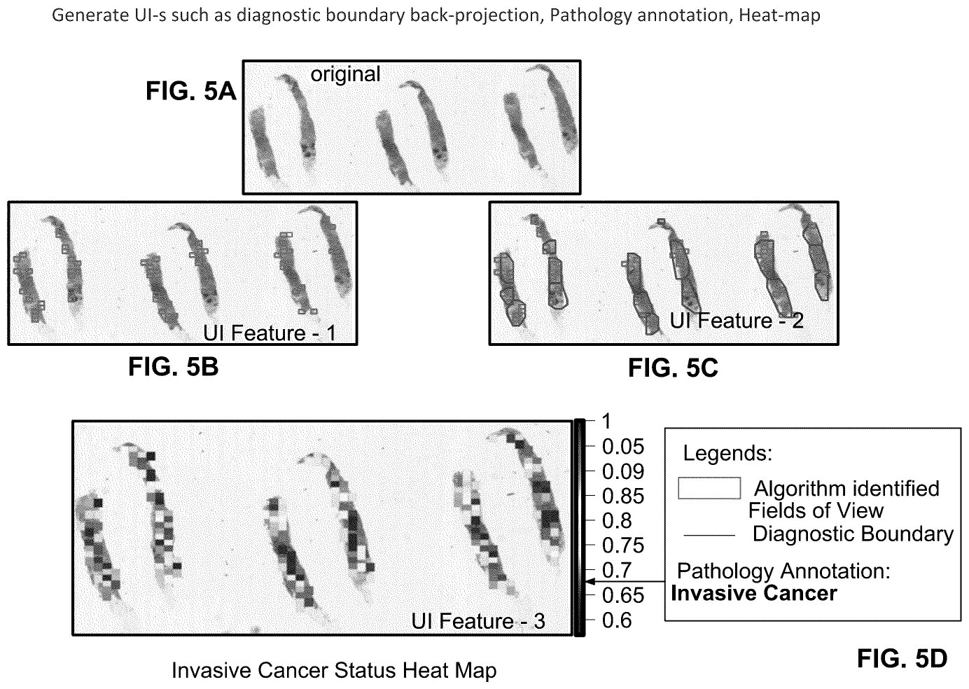

Turning now to Step 3 of the procedure 100 of FIG. 1, a cell cycle deformation function is constructed from a selected subset of values from the vector 108. In certain embodiments, the function includes weights, normalization, and the like. The functions described herein can be constructed from at least a subset of the diagnostic score (which is a vector comprised of multiple values) of a field of view. Each function uses a particular subset of the diagnostic score vector as an input and calculates an output value called an index. The index (referenced in FIG. 1 as “Diagnostic Index”) is then compared against (or mapped to) a corresponding, pre-calibrated reference scale to determine whether the index exceeds a threshold value (or multiple threshold values defining one or multiple ranges on the reference scale) associated with the diagnostic test represented by the function that corresponds to that particular reference scale (e.g., the presence or absence of HER2). The comparison of the diagnostic index to the reference scale (e.g., mapping the index value to the reference scale to see whether it exceeds a threshold value) determines whether the field of view associated with the relevant diagnostic score contains certain properties (e.g., the presence of HER2, cell cycle deregulation, or a property quantified by any of the other seven cell cycle deformation function tests described in detail herein). The result of comparing the index against the reference scale determines whether the field of view associated with that index should be highlighted in the back-projection (see Step 4 of FIG. 1; also see FIG. 5 below). In Step 4 of the procedure of FIG. 1, the fields of view that exceed the threshold are graphically denoted (e.g., highlighted) by rendering a two-dimensional shape (e.g., a polygon) on the image that surrounds all such fields of view. In certain embodiments, user interface features such as heat maps and pathology annotations are then presented, e.g., via an overlay graphically rendered on the image of the biological sample. In the example shown in FIG. 1, a bold polygonal outline identifies the fields of view that exceed the threshold.

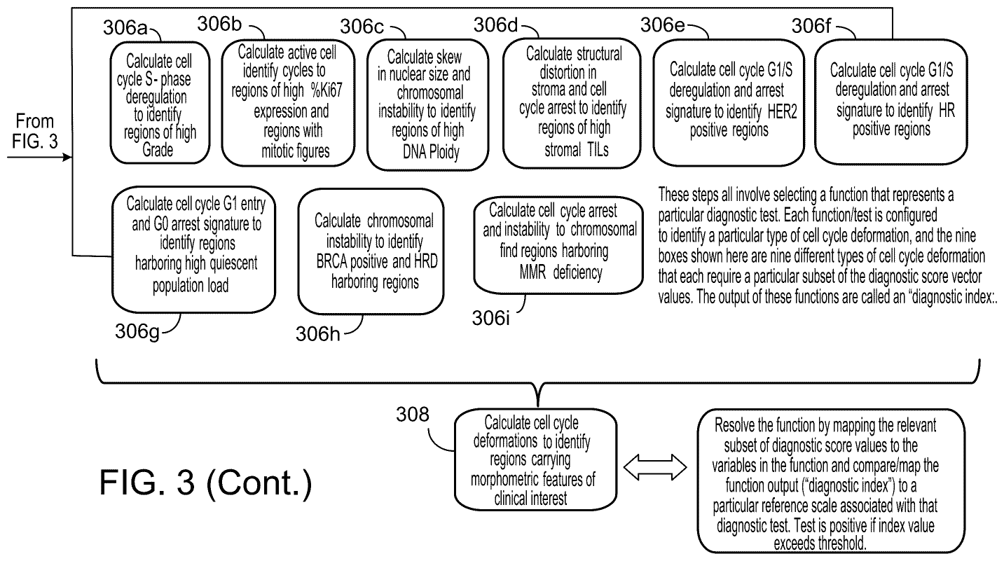

Further to the schematic of FIG. 1, the block flow diagram of FIG. 3 presents a diagnostic procedure 300 for cytological analysis of an image of an in vitro biological sample, according to an illustrative embodiment. Data representing an image of a biological sample is obtained/received (302), where the image comprises an area segmented into one or more subsections (fields of view). The biological sample may include tissue, fluids, or other material. Each subsection has a position identifier, for example, an (x, y) coordinate. Next, respective diagnostic scores are calculated for each of the one or more subsections (304). In certain embodiments, this involves use of one or more cell cycle deformation functions, such as any of the nine specific functions described herein (306a — 306i). The calculated cell cycle deformation for the various subsections of the image are used to identify regions carrying morphometric features of clinical interest (308). For example, the function may be resolved by mapping the relevant subset of diagnostic score values to the variables in the function and comparing/mapping the function output (“diagnostic index”) to a particular reference scale associated with that diagnostic test. The test is positive for a given subsection (field of view) if the index value for that subsection exceeds a given threshold (e.g., a predetermined threshold). Each of a plurality of subsections of the image of the biological sample may be evaluated in this way, and a map of regions of interest may be thusly obtained.

Cancer or any metabolic or inflammatory disease is known to alter various pathways related to key cell cycle proteins, such as cell cycle check-point proteins, DNA-damage pathway proteins, and the like, thus altering overall and various phase specific cell cycle patterns. Often these changes manifest as noticeable phenotype or morphological changes. Collectively, these pattern changes and its manifestations are referred to as cell cycle deformations. Advantageously, it is presently found that the technology described herein can characterize and compute the extent of one or more of these cell cycle deformations from the snapshot image or image data of a whole or a portion of any fixed tissue and population of cells therein (such as H&E stained FFPE tissue images) by capturing the impact these cell cycle deformations on the corresponding/equivalent thermodynamic profile(s) of one or more cells. Disease states (such as invasive cancer of various grades, DNA ploidies etc.) and corresponding molecular (such as ER, PR, HER2, Ki67, BRCA, MMR etc.) or immune (TILs) profiles can be identified if the associated cell cycle deformations are characterized as a continuous scale of vectors or a function thereof and is measured/computed properly. For example, cell cycle deformation like S-phase deregulation can contribute to cancer grade. Higher grades can be associated with higher S-phase deregulation.

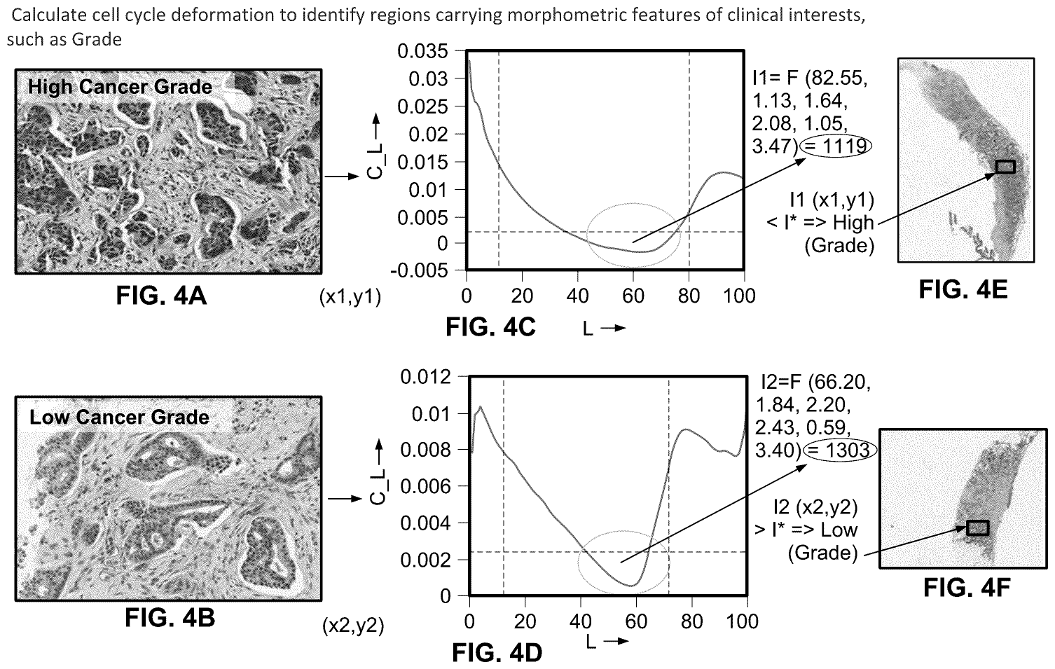

FIG. 4 shows characterization and computation of such deformation (FIG. 4C and FIG. 4D) in terms of a function of numerical vectors extracted from the input image (FIG. 4A and FIG. 4B). Using a threshold on a reference scale (that measures inverse of S-phase deregulation as in our case), the computed values can be assigned to the prediction of high vs low cancer grade (FIG. 4E vs. FIG. 4F). Since this predictive diagnostic value computation (herein from a portion of the full image) is also tagging along the coordinates of whole slide image/image data or an image/image data containing a bigger chunk of the same tissue, these predictions can be presented as a rectangular diagnostic boundary drawn on the original bigger image (containing bigger chunk of the same tissue) at the position specified by the aforementioned coordinates (FIG. 4E, FIG. 4F). All such selected fields of view containing regions of diagnostic interest, each marked by the (red) rectangular boundary (FIG. 5B), can be clustered and enclosed by (blue) bigger polygons (or the convex hull of the coordinates of the selected fields of view) as extended regions of diagnostic interest/diagnostic boundary (FIG. 5C). Similarly, by converting the continuous functions characterizing the cell cycle deformations into a color map (a scale of 0 to 1, binned and mapped to color-schemes), a heat map overlay can be rendered on the original bigger image (containing bigger chunk of the same tissue) using the coordinates (FIG. 5D). All these user interface (UI) features, diagnostic field of view/regions of interest, extended diagnostic boundary/extended regions of diagnostic interest and heat map, along with the prediction of the disease state as the corresponding pathology annotation (for example invasive cancer in FIG. 5) are very useful for pathology review. Currently, it is believed that no other methods (IHC, ISH, FISH, NGS, FACS, IF imaging etc.) can directly compute cellular molecular profiles without using one or multiple molecule specific tags (antibodies, fluorophore etc.). The other methods generally require elaborate laboratory procedures, and many of such methods are purely qualitative or semi-quantitative.

To briefly summarize, FIG. 4A and FIG. 4B depict an input image/image data/field of view containing high-grade and low-grade cancer respectively. FIG. 4C and FIG. 4D depict functional computation of corresponding cell cycle deformation (such as S-phase deregulation) as diagnostic index, using a portion of vector array numerically characterizing the cellular/biological and biologic information contained in the Field of view, to predict the extent of the disease state (such as high or low cancer grade or cancer grade 3 (high), grade 2 (high) and grade 1 (low)). The curves represent corresponding visualization of the respective deformations in 2D (C_L,L) space, where (C_L,L) are quantities described throughout this specification. FIG. 4E and FIG. 4F show identification and annotation of regions of interest on the bigger/whole slide image (containing larger portion of the same tissue or the entire tissue) using the score as a function of (x,y) coordinates.

FIG. 5A depicts an input whole slide image; FIG. 5B and FIG. 5C depict a whole slide image annotated with fields of views containing regions of diagnostic interest (labelled red), and diagnostic boundary containing extended regions of interest (labelled blue). In this case, the primary or extended regions of diagnostic index is for the pathology annotation pointing to tissue regions containing invasive cancer. FIG. 5D depicts a heat map with color bar corresponding to the degree of invasive cancer at any (x,y) point on the whole slide image. As shown in FIG. 1 and FIG. 3, the illustrative diagnostic processes use the following twelve functions for predicting certain diagnostic entities as defined below in Table 1. Note that, the variables referenced in the last column are described throughout this specification.

TABLE 1
Different types of measured cell cycle deformations and corresponding functions

| | | | Corresponding Cell Cycle Functions: |
|---|---|---|---|
| Function | Diagnostic | Surrogate Biological | Diagnostic Vector Mapping of |
| Index | Entity | Signature | Thermodynamic Decision Parameters |
| 1 | Cancer | Deregulation of cell | Function 1: Shape (<C>) junction |
| | histological | cycle DNA synthesis | curvature variance |
| | grade, Mitotic | phase (S- Phase) | |
| | Figures | | |
| 2 | Ki67 status, | Cell cycle active | Function 2: (Skew in C_L-initial) × |
| | Mitotic | phase length; M- | (Skew in C_L-max) |
| | figures | phase activity | |
| 3 | DNA ploidy | Skew in nuclear size | Function 3: Percentage of tissue arca |
| | | and chromosomal | with high skew in (C_L)max |
| | | instability | |
| 4 | Stromal TILs | Structural distortion | Function 4: Shape(<C>) deviation |
| | status | in stroma and overall | |
| | | measure of cell cycle | |
| | | arrest | |
| 5 | HER2 status | Cell cycle G1/S | Function 5: Shape (<C_L>) |
| | | deregulation and | divergence |
| | | arrest signature | |
| 6 | HR | Cell cycle G1/S | Function 6: Shape (<C>) divergence |
| | (combined | deregulation and | |
| | ER, PR) | arrest | |
| | status | signature | |
| 7 | Quiescent | Cell cycle G1 entry | Function 7: (<C>max-<C>min) × |
| | Population | and G0 arrest | (Skew in |
| | Load | signature | <C>max) |
| 8 | BRCA | Chromosomal | Function 8: Jump in shape (<C>) |
| | mutation and | instability | bound |
| | HRD status | | |
| 9 | MMR | Cell cycle arrest and | Function 9: Shape (<C>) section slope |
| | deficiency | chromosomal | Ructuation |
| | status | instability | |
| 10 | BRAF and/or | Cell cycle G1 entry | Function 10: Shape |
| | NRAS | and G1/S | (<C>max, <C>min, <C>) divergence |
| | mutation | deregulation | |
| | status in | signature | |
| | Malignant | | |
| | Melanoma | | |
| 11 | Degree of | Cell cycle G1/S | Function 11: Shape (<C_L>, <C>) |
| | Dysplasia | deregulation | divergence |
| | | signature | |
| 12 | Degree of | Cell cycle G2/M | Function 12: Shape (<C_L>, <C>max) |
| | Dysplasia | deregulation | divergence & (<C>max-<C>min) shift |
| | | signature | |

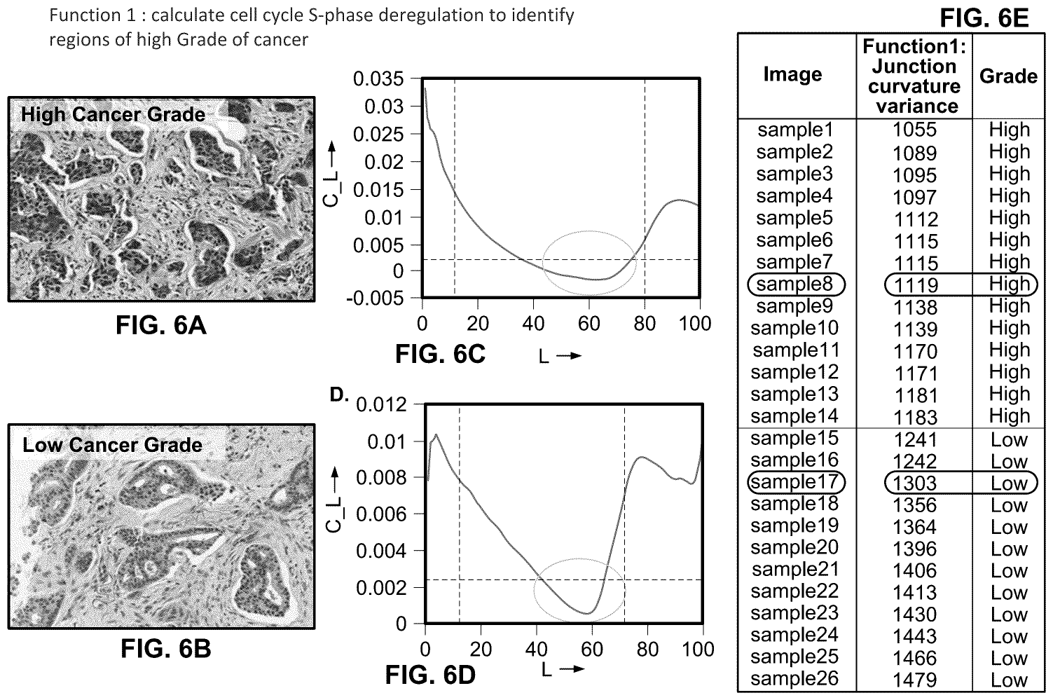

Function 1: Cell cycle S-phase deregulation can contribute to cancer grade. The higher the extent of deregulation is, the higher is the risk of harboring damaged DNA, hence, the risk of developing higher grade cancers. The cancer grade, which, in certain embodiments, is an important part of the pathology diagnosis, is a measure for pathologists to assess how aggressive a cancer can be. FIG. 3 and FIG. 4 present an illustrative general methodology of how any such deregulation present in an image/portion of an image/image data, can be first computed as diagnostic scores (comprising vector array numerically characterizing the cellular/biological and biologic information contained in the input image/image-data) from pixel data (nuclear area and contrast), subsequent mapping of a portion of the diagnostic score to a specific diagnostic index value and its back-projected on the whole slide image using original image (x,y) coordinates. FIG. 6 presents a specific example of how S-phase deregulation capturing cancer grade can be computed by the function F1, which essentially measures the inverse of such deregulation, such that low cancer grade (grade 1) can be identified above a certain threshold and the higher grades (grade 2 and grade 3) can be identified below the threshold. Note that, multiple thresholds are used to separate high grade cancer into grade 2 and grade 3. A unique aspect of the construction of Function 1 is that in the (C_L, L) space, Function 1 demonstrates visibly differential variance of the curvature indicative of such deregulation.

FIG. 6A and FIG. 6B depict input image/image data/field of view containing high-grade and low-grade cancer respectively. FIG. 6C and FIG. 6D depict the functional computation of corresponding cell cycle deformation manifested in S-phase deregulation as a diagnostic index, using a portion of vector array numerically characterizing the cellular/biological and biologic information contained in the field of view, thus predicting the extent of the cancer grade (such as high/low or cancer grade 3 (high), grade 2 (high) and grade 1 (low)). The curves represent corresponding visualization of the respective deformations in 2D (C_L,L) space. FIG. 6E is a table that demonstrates determination of high vs low cancer grade using Function 1 below and above certain threshold.

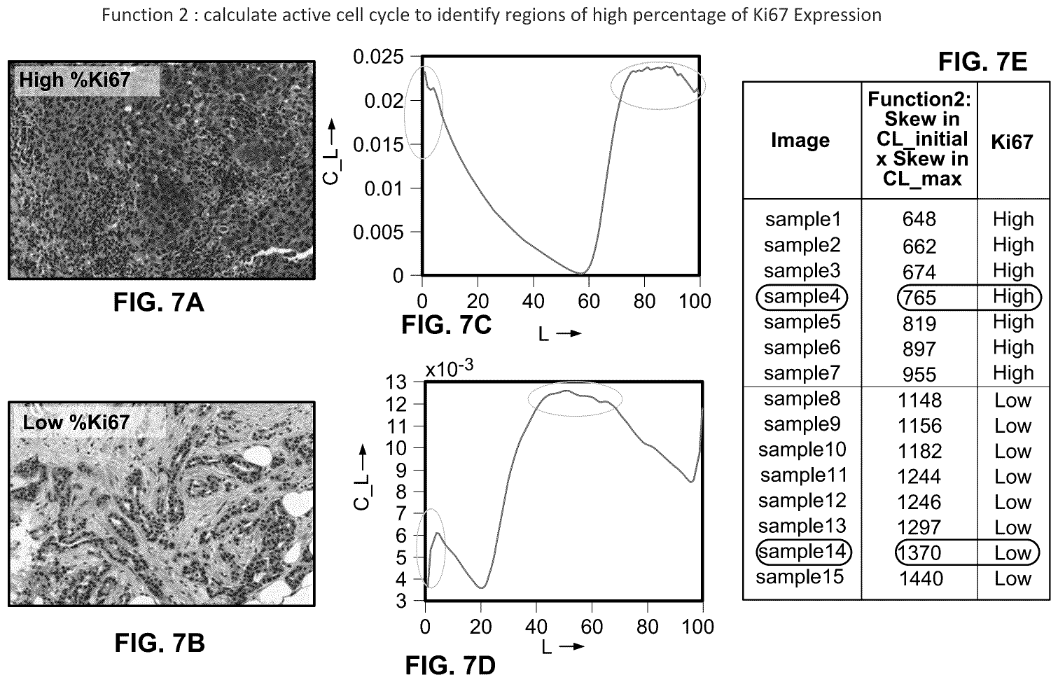

Function 2: Ki67 protein is a marker for cellular activity and proliferation. Ki-67 protein is present during all active phases of the cell cycle (G1, S, G2, and mitosis), but is absent in resting/quiescent cells (G0). The fraction/percentage of Ki67-positive tumor cells may be correlated with the clinical course of cancer. For example, in breast cancer, Ki67 identifies a high proliferative subset of patients with ER-positive breast cancer who derive greater benefit from adjuvant chemotherapy. In essence, percentage Ki67 marker, like cancer grade, measures overall aggressiveness of the tumor in terms of tumor growth. Thus, cell cycle active phase duration and its deformation is naturally implicated in the determination of Ki67-expression. FIG. 3 and FIG. 4 explain a methodology of how any such deformation present in an image/portion of an image/image data can be first computed as diagnostic scores (comprising vector array numerically characterizing the cellular/biological and biologic information contained in the input image/image-data) from pixel data (nuclear area and contrast), subsequent mapping of a portion of the diagnostic score to a specific diagnostic index value and its back- projected on the whole slide image using original image (x,y) coordinates. FIG. 7 depicts a specific example of how deformation in cell cycle active phase duration capturing % Ki67 expression can be computed by the Function 2, which essentially measures the inverse of M-phase specific cellular activity affecting the cell cycle entry pattern, such that low % Ki67 can be identified above a certain threshold and high % Ki67 can be identified below the threshold. A unique aspect of the construction of Function 2 is that in the (C_L, L) space, Function 2 demonstrates visibly differential entry and exit point signature indicative of such deformation.

FIG. 7A and FIG. 7B depict input image/image data/field of view of cancerous tissue containing high and low % Ki67 expression, respectively. FIG. 7C and FIG. 7D depict functional computation of corresponding deformation in cell cycle active phase length as the novel diagnostic index, using a portion of vector array numerically characterizing the cellular/biological and biologic information contained in the field of view, thus predicting the extent of the of high and low % Ki67 expression. The curves represent corresponding visualization of the respective deformations in 2D (C_L,L) space. FIG. 7E is a table that demonstrates determination of high vs low % Ki67 expression status of cancer using Function 2, below and above certain threshold.

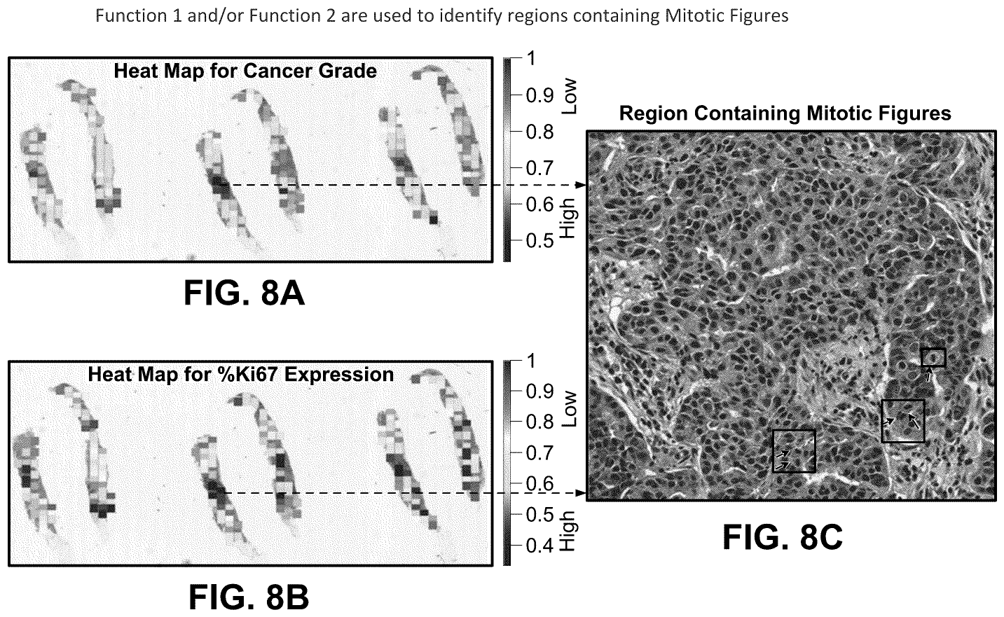

A corollary/variant of Function 1 and Function 2: Both high grade and high % Ki67 expression may be associated with high mitotic figures. A mitotic figure is a cell that is in the process of dividing to create two new cells. This process is called mitosis. Pathologists can see mitotic figures when they look at tissue under the microscope. Also, pathologists routinely report and score the number of mitotic figures present in a tissue sample for the purpose of calculating cancer grade. Such regions of interests containing mitotic figures can be identified from a heat map of the Function 1 or Function 2 by pointing to the region of high grade or high % ki67 expression, as shown in FIG. 8, for example.

FIG. 8A depicts a heat map of cancer grade using the Function 1 value as a function of (x,y) coordinates on the whole slide image. FIG. 8B depicts a heat map of % ki67 Expression using the Function 2 value as a function of (x,y) coordinates on the whole slide image. FIG. 8C depicts an example blown up region of the whole slide image containing mitotic figures, which is identified by looking for high grade or high % ki67 expression regions from the heat maps in FIG. 8A and FIG. 8B.

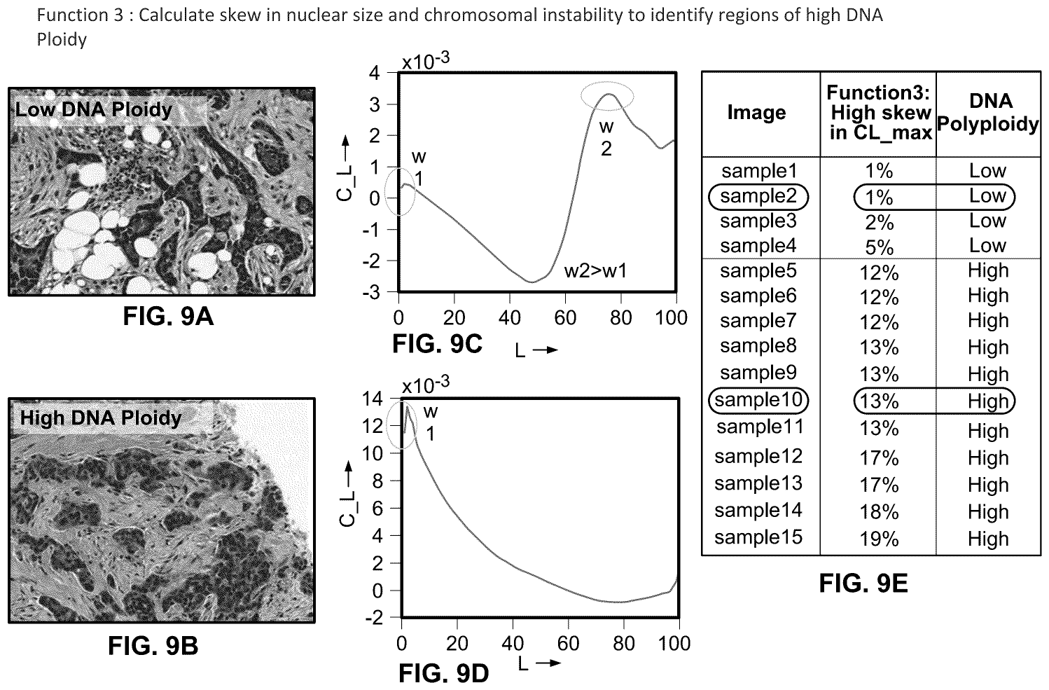

Function 3: The ploidy of cancer cells refers to the amount of DNA they contain. If there's a normal amount of DNA in the cells, they are said to be diploid. These cancers tend to grow and spread more slowly. If the amount of DNA is abnormal (numerical or structural, depending on whether whole chromosomes or portions of chromosomes are gained or lost) then the cells are called aneuploid. If the cells contain more than two complete sets of chromosomes, but always contain an exact multiple of the haploid number, so the chromosomes remain balanced, then they are called polyploid. A good correlation exists between DNA ploidy and histologic grade, and DNA ploidy adds clinically useful predictive information for some patients. Most low-stage tumors are diploid and high-stage tumors are non-diploid. Cells harboring high DNA ploidy exhibit highly skewed/disorganized nuclear and chromatin material. Here, by DNA ploidy, we refer to any such deviation from the diploid status. FIG. 9 presents a specific example of how the skew in nuclear size and chromosomal instability characterizing DNA ploidy can be computed by the Function 3, which essentially measures the cellular chromosomal instability affecting the cell cycle exit patterns, such that the high DNA ploidy can be identified above a certain threshold and the low DNA ploidy can be identified below the threshold. A unique aspect of the construction of Function 3 is that in the (C_L, L) space, Function 3 demonstrates visibly differential exit signature indicative of such deformation.

FIG. 9A and FIG. 9B present an image/image data/field of view of cancerous tissue harboring low and high DNA ploidy respectively. FIG. 9C and FIG. 9D demonstrate functional computation of corresponding skew in nuclear size and chromosomal instability as the novel diagnostic index, using a portion of vector array numerically characterizing the cellular/biological and biologic information contained in the field of view, thus predicting the extent of the DNA ploidy. The curves represent corresponding visualization of the respective deformations in 2D (C_L,L) space. FIG. 9E is a table that demonstrates determination of low vs high DNA ploidy status of cancer using the Function 3, below and above certain threshold.

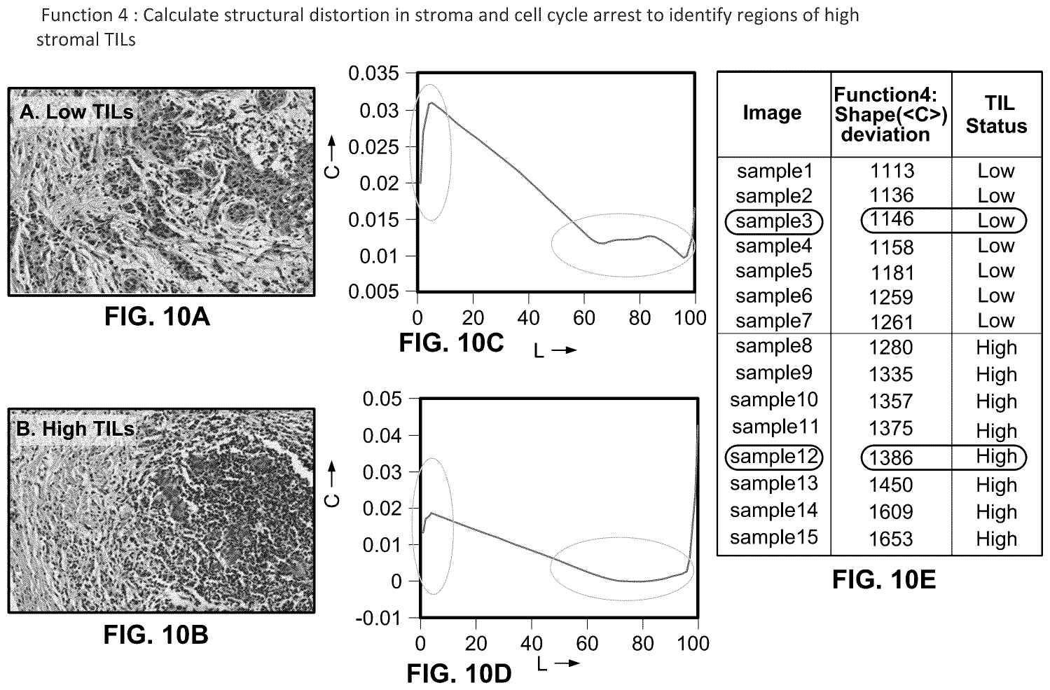

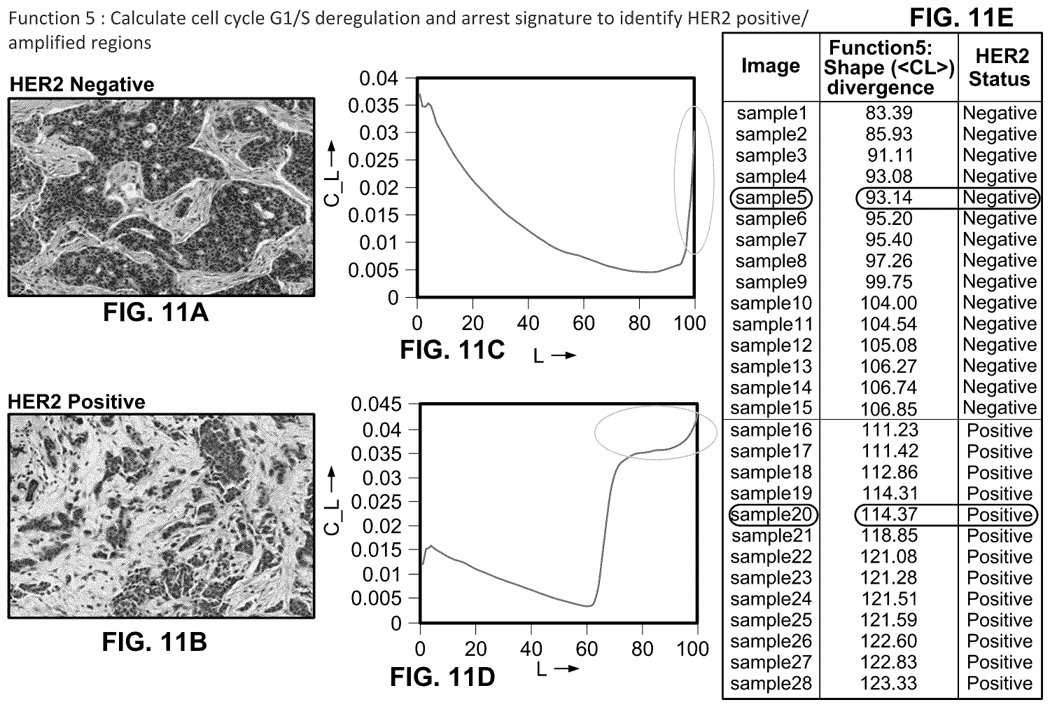

Function 4: Tumor infiltrating lymphocytes (TILs) refer to all lymphocytic cell populations that have invaded the tumor tissue. TILs have been described in a number of solid tumors, including breast cancer, and are emerging as an important biomarker in predicting the efficacy and outcome of treatment. Pathologists can see TILS under microscope. FIG. 10 shows a specific example of how the structural distortion in stroma and cell cycle arrest characterizing stromal TILS expression can be computed by the Function 4, which essentially measures the stromal geometry and cellular activity deviation, such that the low stromal TILS expression can be identified above a certain threshold and the high stromal TILs expression can be identified below the threshold. A unique aspect of the construction of Function 4 is that in the (C_L, L) space, Function 4 demonstrates visibly differential shapes indicative of such deformation.

FIG. 10A and FIG. 10B depict an input image/image data/field of view of cancerous tissue harboring low and high stromal TILs respectively. FIG. 10C and FIG. 10D show functional computation of corresponding structural distortion in stroma and overall measure of cell cycle arrest as the novel diagnostic index, using a portion of vector array numerically characterizing the cellular/biological and biologic information contained in the field of view, thus predicting the extent of the stromal TILs expression. The curves represent corresponding visualization of the respective deformations in 2D (C_L,L) space. FIG. 10E is a table that demonstrates determination of low vs high stromal TILs status using the Function 4, below and above certain threshold.

Function 5: HER2/human epidermal growth factor receptor 2 is a protein which promotes the growth of cancer cells. In about 1 of every 5 breast cancers, the cancer cells have extra copies of the gene that makes the HER2 protein. HER2 amplification/overexpression test is a routine reflex-test done to measure the prognosis of the cancer. HER2 positive cancers can have poor prognosis and often metastasize to brain. FIG. 11 shows a specific example of how the cell cycle G1/S transition deregulation and arrest signature characterizing HER2 amplification/overexpression can be computed by the Function 5, which essentially measures the G1/S transition specific cellular activity deviation, such that the positive HER2 status (amplified/overexpressed) can be identified above a certain threshold and the negative HER2 status (nonamplified/non-overexpressed) can be identified below the threshold. A unique aspect of the construction of Function 5 is that in the (C_L, L) space, the function demonstrates visibly differential shapes indicative of such deformation.

FIG. 11A and FIG. 11B depict an image/image data/field of view of cancerous tissue with presence/absence of HER2 amplification/overexpression (referred to as positive/negative) respectively. FIG. 11C and FIG. 11D demonstrate functional computation of corresponding cell cycle G1/S deregulation and arrest signature as the novel diagnostic index, using a portion of vector array numerically characterizing the cellular/biological and biologic information contained in the field of view, thus predicting the extent of HER2 amplification/overexpression status. The curves represent corresponding visualization of the respective deformations in 2D (C_L,L) space. FIG. 11E is a table that demonstrates determination of positive vs. negative HER2 status using the Function 5, below and above a certain threshold.

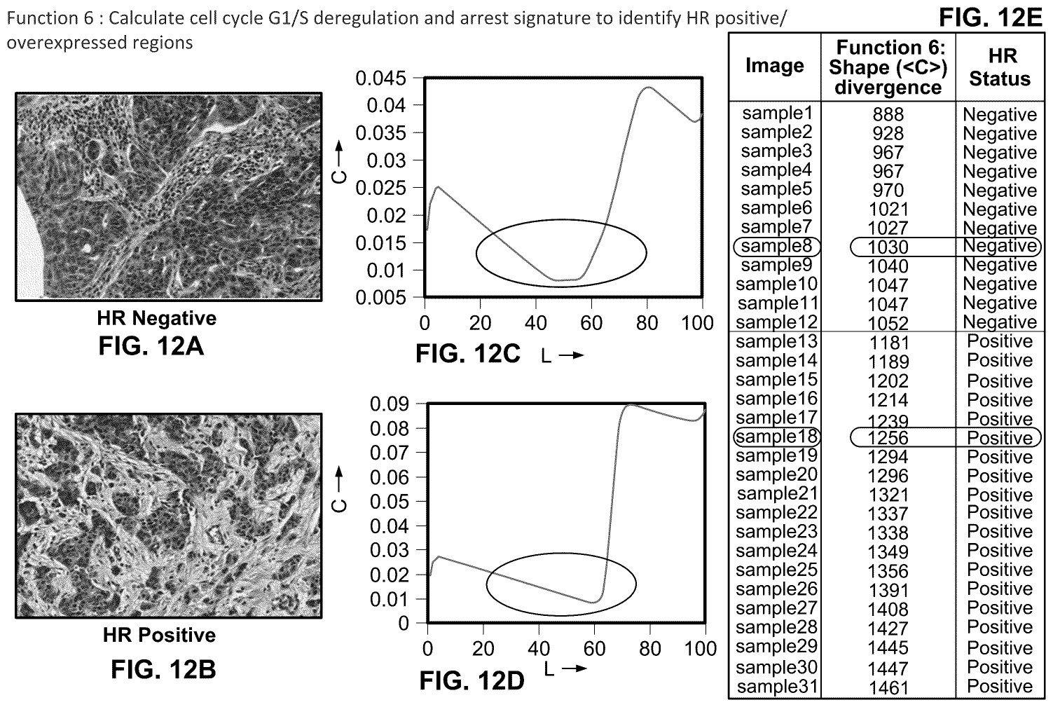

Function 6: Estrogen and progesterone receptors are found in breast cancer cells that depend on estrogen and related hormones to grow. Patients diagnosed with invasive breast cancer or a breast cancer recurrence routinely have their tumors tested for estrogen (ER) and progesterone (PR) receptors which together is referred to as hormone receptors (HR). FIG. 12 shows a specific example of how the cell cycle G1/S transition deregulation and arrest signature characterizing HR overexpression can be computed by the Function 6, which essentially measures the G1/S transition specific cellular activity deviation, such that the positive HR status (overexpressed) can be identified above a certain threshold and the negative HR status (non- overexpressed) can be identified below the threshold. A unique aspect of the construction of Function 6 is that in the (C_L, L) space, Function 6 demonstrates visibly differential shapes indicative of such deformation.

FIG. 12A and FIG. 12B show an input image/image data/field of view of cancerous tissue with absence/presence of HR overexpression (referred to as negative/positive) respectively. FIG. 12C and FIG. 12D demonstrate functional computation of corresponding cell cycle G1/S deregulation and arrest signature as the novel diagnostic index, using a portion of vector array numerically characterizing the cellular/biological and biologic information contained in the Field of view, thus predicting the extent of HR overexpression status. The curves represent corresponding visualization of the respective deformations in 2D (C_L, L) space. FIG. 12E is a table that demonstrates determination of negative vs. positive HR status using the Function 6, below and above certain threshold.

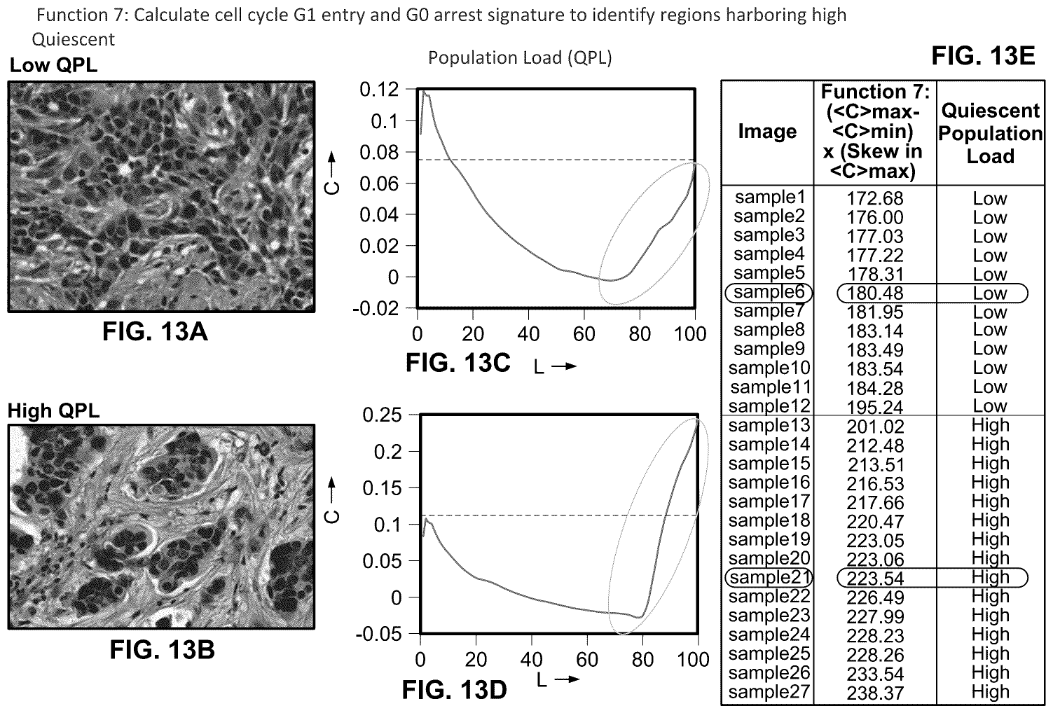

Function 7: Quiescence is a state of reversible proliferative arrest in which cells are not actively dividing and yet retain the capacity to reenter the cell cycle upon receiving an appropriate stimulus. Quiescent population of cells are believed to be resting in G0 phase of cell cycle and can render cancer recurrence one chemotherapy or other targeted therapies are withdrawn. Thus, in certain embodiments, a measure of such quiescent population load (QPL) may be an important marker for evaluating prognosis or treatment efficacy. FIG. 13 shows a specific example of how the cell cycle G1 entry and G0 arrest signature characterizing QPL can be computed by the Function 7, which essentially measures the G1 specific cellular activity deviation, such that the high QPL status can be identified above a certain threshold and the low QPL status (non-amplified/non-overexpressed) can be identified below the threshold. A unique aspect of the construction of Function 7 is that in the (C_L, L) space, Function 7 demonstrates visibly differential shape indicative of such deformation.

FIG. 13A and FIG. 13B show an image/image data/field of view of cancerous tissue with low vs high quiescent population load (QPL). FIG. 13C and FIG. 13D demonstrate functional computation of corresponding cell cycle G1 entry and G0 arrest signature as the novel diagnostic index, using a portion of vector array numerically characterizing the cellular/biological and biologic information contained in the Field of view, thus predicting the extent of quiescent population present in the tissue. The curves represent corresponding visualization of the respective deformations in 2D (C_L,L) space. FIG. 13E is a table that demonstrates determination of low vs high QPL using the Function 7, below and above certain threshold.

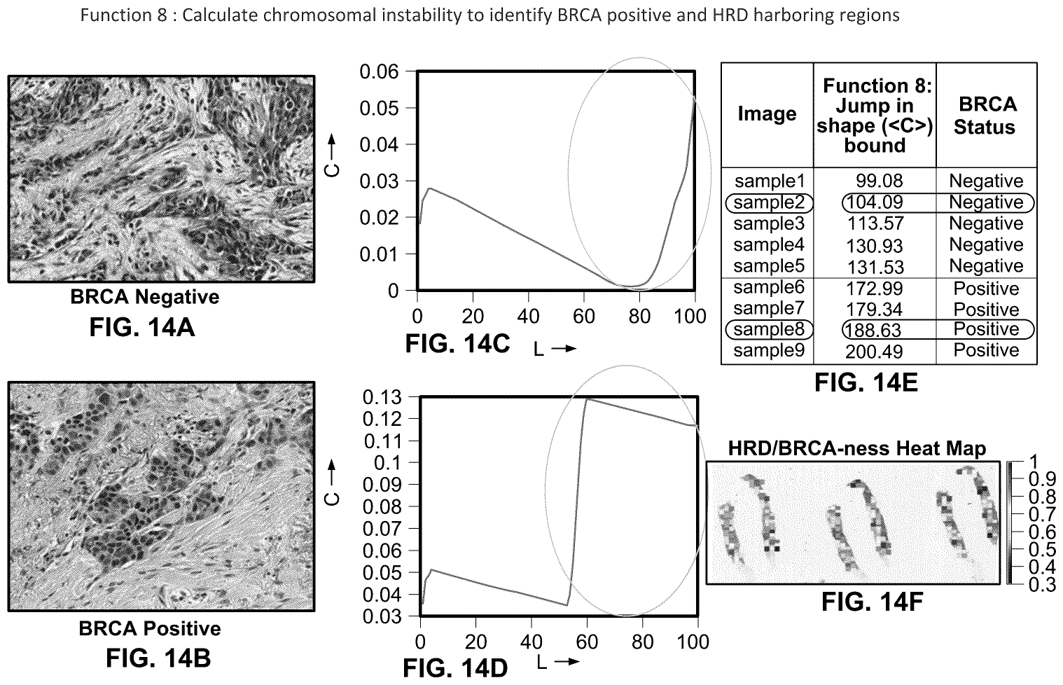

Function 8: “BRCA” is an abbreviation for “BReast CAncer gene.” BRCA1 and BRCA2 are two different genes that have been found to impact a person's chances of developing breast cancer if harboring certain mutations. Homologous recombination is a highly accurate DNA repair mechanism. When BRCA genes are mutated or the tumor harbors other vulnerabilities damaging the DNA repair mechanisms, then the condition is identified as Homologous Recombination Deficiency. If HRD is present then cancer cells are particularly vulnerable to certain targeted therapies, such as PARP-inhibitor therapy. Thus, in certain embodiments, identifying presence of BRCA mutation or HRD is important for selection of appropriate treatment. FIG. 14 presents a specific example of how the chromosomal instability signature characterizing BRCA mutation and HRD status can be computed by the Function 8, which essentially measures the divergent shift in cellular activity, such that the positive BRCA status (mutated) can be identified above a certain threshold and the negative BRCA status (non- mutated) can be identified below the threshold. A unique aspect of the construction of Function 8 is that in the (C_L, L) space, Function 8 demonstrates visibly differential shapes indicative of such deformation. HRD is computed from the local fluctuation of Function 8.

FIG. 14A and FIG. 14B show an input image/image data/field of view of cancerous tissue with wild type/ mutated BRCA1 or BRCA2 gene (referred to as BRCA negative/positive) respectively. FIG. 14C and FIG. 14D demonstrate functional computation of corresponding chromosomal instability signature as the novel diagnostic index, using a portion of vector array numerically characterizing the cellular/biological and biologic information contained in the field of view, thus predicting the extent of BRCA mutation status. The curves represent corresponding visualization of the respective deformations in 2D (C_L,L) space. FIG. 14E is a table that demonstrates determination of BRCA negative vs positive status using the Function 8, below and above certain threshold. FIG. 14F demonstrates how HRD status may be computed from the local fluctuation/variability of the Function 8 and the HRD status is shown as a heat map as a function of (x,y) coordinates.

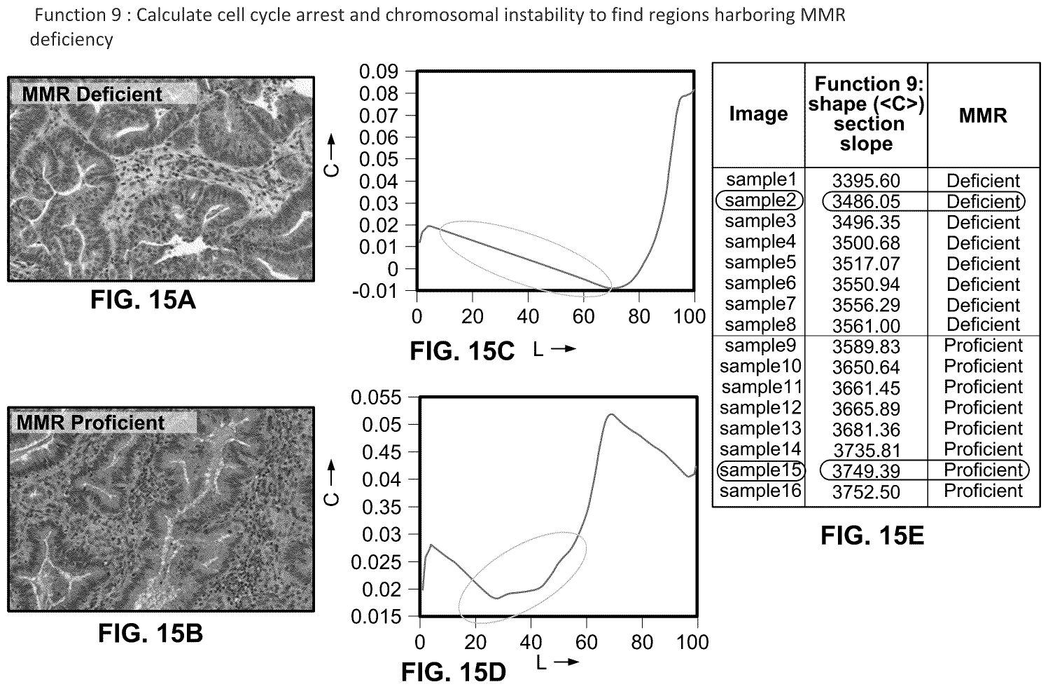

Function 9: Mismatch repair (MMR) deficient cells usually have many DNA mutations, which may lead to cancer. The presence of functional tumour MMR deficiency can be assessed by either tumor microsatellite instability (MSI) testing. MMR deficiency is most common in colorectal cancer, other types of gastrointestinal cancer, and endometrial cancer, but it may also be found in cancers of the breast, prostate, bladder, and thyroid. FIG. 15 presents a specific example of how the cell cycle arrest and chromosomal instability signature characterizing MMR deficiency status can be computed by the Function 9, which essentially measures the fluctuation in the shape representing the cellular activity, such that the MMR proficient status can be identified above a certain threshold and the MMR deficient status can be identified below the threshold. A unique aspect of the construction of Function 8 is that in the (C_L, L) space, Function 9 demonstrates visibly differential shapes indicative of such deformation.

FIG. 15A and FIG. 15B show an input image/image data/field of view of cancerous tissue with MMR deficient vs proficient status. FIG. 15C and FIG. 15D demonstrate functional computation of corresponding cell cycle arrest and chromosomal instability signature as the novel diagnostic index, using a portion of vector array numerically characterizing the cellular/biological and biologic information contained in the Field of view, thus predicting the extent of MMR deficiency status. The curves represent corresponding visualization of the respective deformations in 2D (C_L,L) space. FIG. 15E is a table that demonstrates determination of deficient vs proficient MMR status using Function 9, below and above a certain threshold.

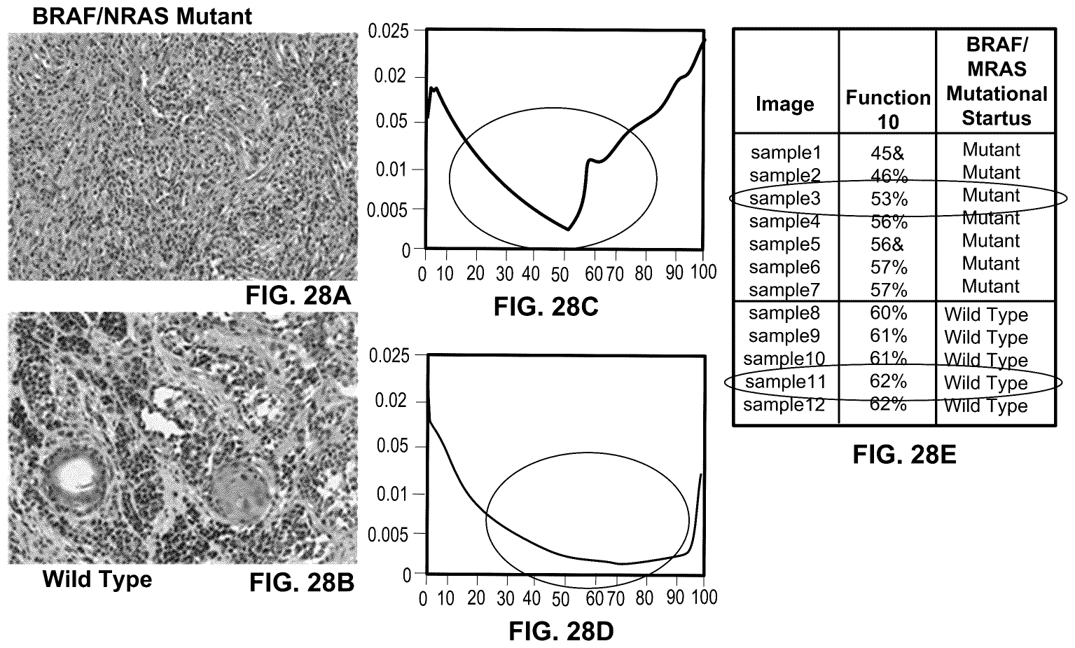

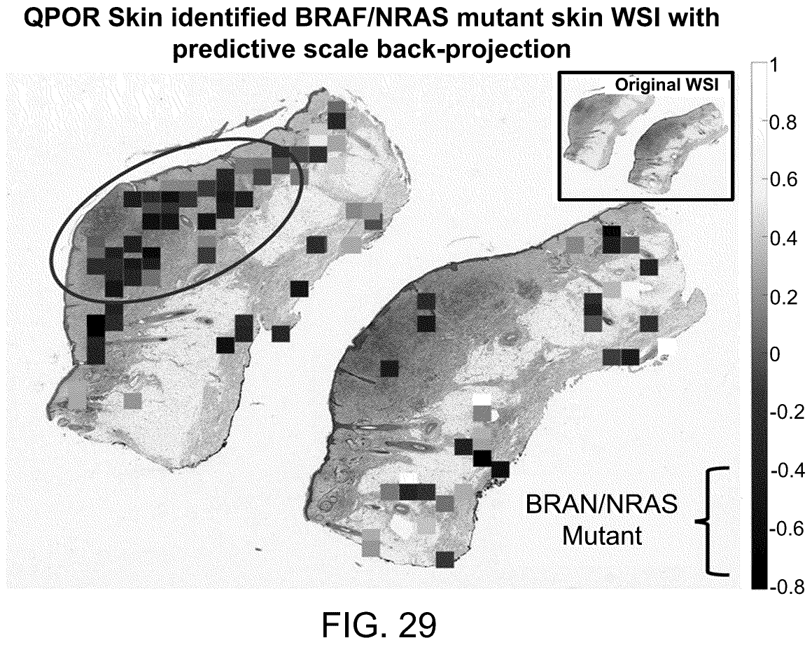

Function 10: Melanoma is an aggressive malignancy originating from melanocytes of the skin with a high tendency to metastasize. Activating mutations in the oncogenes BRAF and NRAS are very common (e.g., almost 50% prevalence of BRAF 600 mutation and 20% prevalence of NRAS mutation) in malignant Melanomas. Identification of the presence of BRAF and/or NRAS mutations in Melanoma specimen offers both prognostic and therapeutic guidance. Both of these mutations affect mitogen-activated protein kinase (MAPK) pathway contributing to enhanced tumor growth and promoting disease progression. From the cell cycle point of view this can directly translate into highly deregulated cell cycle, particularly in cell cycle entry and G1/S transition. FIG. 28 presents a specific example of how the deregulation in cell cycle G1 phase entry and G1/S transition phase characterizing BRAF and/or NRAS mutation can be computed from the Function 10, which essentially measures the inverse of the collective strength of those deformations such that the BRAF and/or NRAS mutant status is identified above a threshold and the wild-type (generic) status is identified below the threshold. A unique aspect of the construction of Function 10 is that in the (C_L, L) space, Function 10 demonstrates visibly differential divergence signature indicative of such deformation. The regions of interests containing BRAF and/or NRAS mutations can be identified from a heat map of the Function 10 as explained with reference to FIG. 29.

FIG. 28A. and FIG. 28B show an input image/image data/field of view of cancerous tissue (in this case malignant melanoma) harboring BRAF/NRAS mutant and wild-type respectively. FIG. 28C and FIG. 28D demonstrate functional computation of corresponding cell cycle G1 entry and G1/S deformation as the novel diagnostic index, using a portion of a vector array numerically characterizing the cellular/biological and biologic information contained in the field of view, thus predicting the extent of the BRAF/NRAS mutation status. The curves represent corresponding visualization of the respective deformations in 2D (C_L,L) space. FIG. 28E is a table that demonstrates determination of BRAF/NRAS mutant vs wild-type status of cancer (here malignant melanoma) using the Function 10, below and above certain threshold.

FIG. 29 shows a heat map of BRAF/NRAS mutation status in malignant melanoma using the Function 10 value as a function of (x,y) coordinates on the whole slide image (original image is shown inset).

Function 11 and Function 12: Barrett's esophagus is a pathology of esophagus involving complication of gastroesophageal reflux disease (GERD) and is associated with an increased risk of esophageal adenocarcinoma (cancer of esophagus). Roughly 30 million people in North America have GERD, which is the most common chronic gastrointestinal disease. About 5 percent of patients with chronic GERD or inflammation of the esophagus are known to develop Barrett's esophagus. GERD is a condition in which the stomach contents, including acid, reflux into the esophagus. Barrett's esophagus is a consequence of chronic GERD which can occur when the lining of the esophagus (including the squamous mucosa) heals abnormally and changes from skin cells (squamous cells) to cells that have characteristics of intestinal cells (e.g., specialized columnar cells with intestinal metaplasia). Because of their increased risk of developing cancer, patients diagnosed with Barrett's esophagus may require on-going surveillance, such as long-term. follow-up/repeat esophagogastroduodenoscopy. If precancerous cells (dysplasia) are diagnosed in Barrett's esophagus, then endoscopic treatment may be recommended as a safe and effective for preventing progression to cancer. Detection of dysplasia and the degree/extent of dysplasia are crucial prognostic factors in Barrett's esophagus. From the cell cycle point of view, occurrence of dysplasia can stem from a lack of tumor suppression effects which directly translate into a highly deregulated cell cycle, particularly in cell cycle G1/S and G2/M transitions.

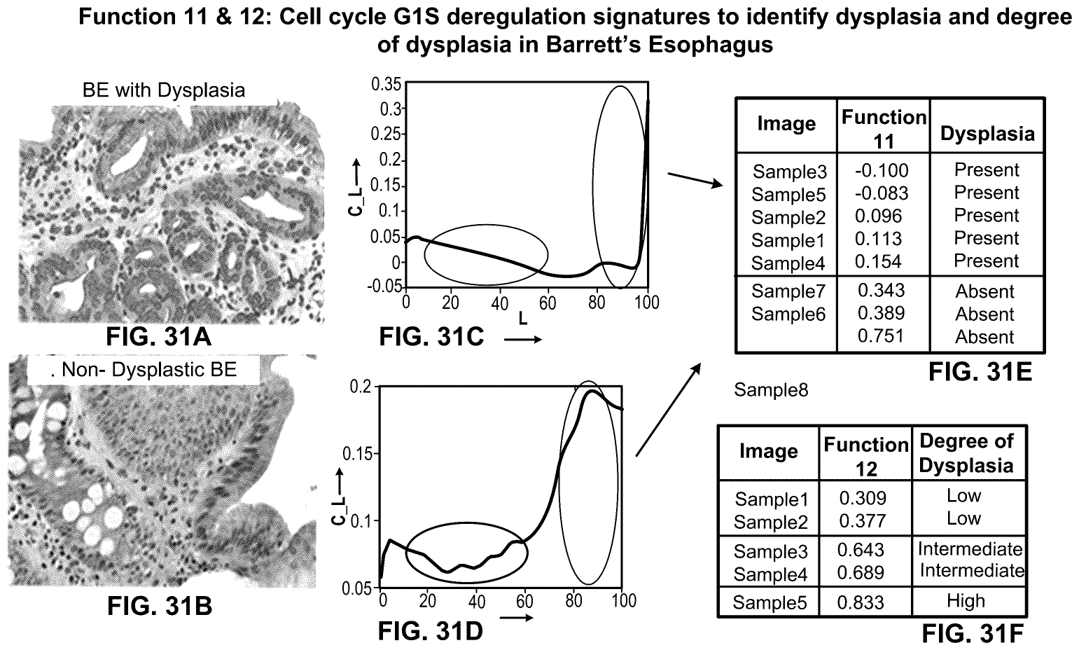

FIG. 31 presents a specific example of computing deregulation in cell cycle G1/S and G2/M transition phases (e.g., characterizing presence/absence of dysplasia and the degree of dysplasia status) using Function 11 and Function 12 respectively. Function 11 and Function 12 measure an inverse and a direct correlation of the strength of G1/S and G2/M deformations respectively. In particular, the presence of the dysplasia can be identified above a threshold of Function 11, and the non-presence of dysplasia can be identified below the threshold of the Function 11. The degree of dysplasia (e.g., low, intermediate, or high) is aligned with certain ranges of thresholds along increasing values of the Function 12. A unique aspect of the construction of Function 11 and Function 12 is that, in (C_L,L) space, Function 11 and Function 12 demonstrate visibly differential divergence signatures indicative of G1/S and G2/M deformations.

FIG. 31A and FIG. 31B show an input image/image data/field of view of pre-malignant esophageal tissue (in this case, dysplastic Barret' s esophagus) and benign esophageal tissue (in this case, non-dysplastic Barrett's esophagus) respectively. FIG. 31C and FIG. 31D demonstrate functional computation of corresponding cell cycle G1/S and G2/M deregulation as novel diagnostic indices (using a portion of a vector array numerically characterizing the cellular/biological and biologic information contained in the field of view), thus predicting: (i) the presence or absence of dysplasia (e.g., using Function 11), and (ii) the degree/extent of dysplasia (e.g., using Function 12). The curves shown in FIG. 31C and FIG. 31D represent corresponding visualizations of respective deformations in 2D (C_L,L) space. FIG. 31E is a table that demonstrates determination of the presence/absence of dysplasia in the esophagus based on whether Function 11 is below or above a certain threshold. FIG. 31F is a table that demonstrates determination of degree/extent of dysplasia in a dysplastic esophagus using Function 12 for certain ranges of thresholds.

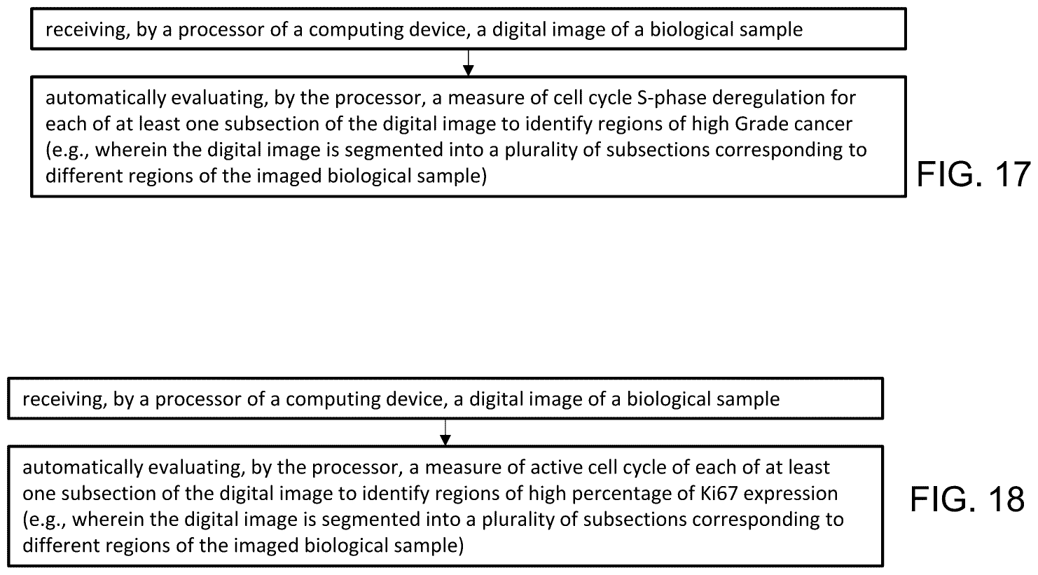

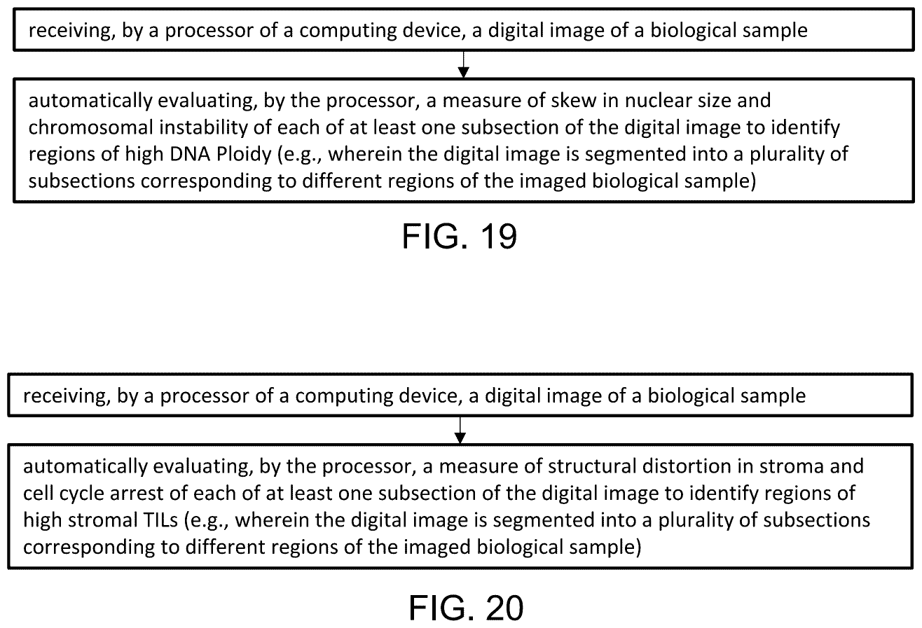

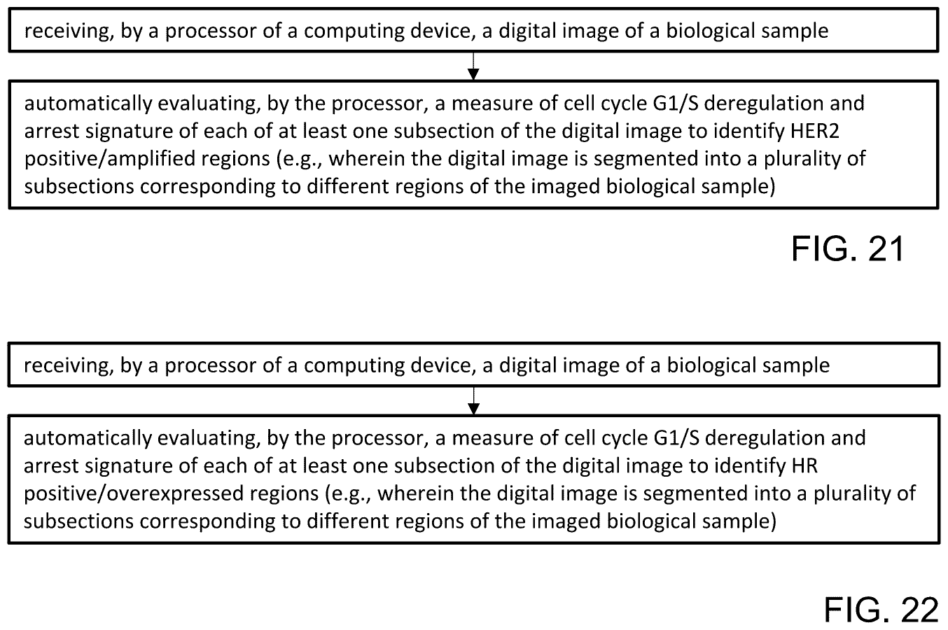

FIG. 17-FIG. 24 show illustrative methods involving computation of the measured cell cycle deformations and corresponding functions of Table 1 described above—particularly, Functions 1-9.

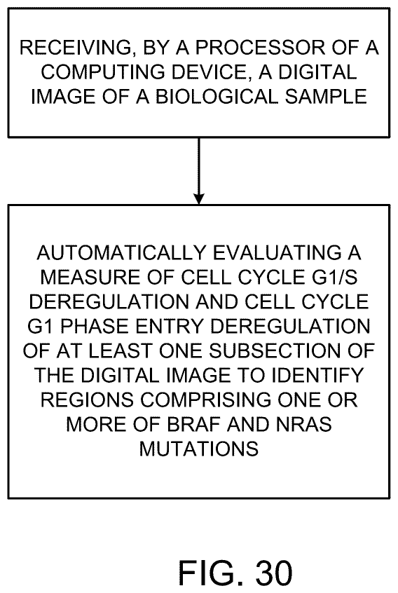

In certain embodiments, a network environment as depicted in FIG. 30 of U.S. Pat. No. 10,535,434 and described therein (e.g., at col. 23) may be used in the methods and systems described herein. Furthermore, in certain embodiments, the computing devices depicted in FIG. 31 of U.S. Pat. No. 10,535,434 and described therein (e.g., at cols. 23-27) may be used in the methods and systems described herein.

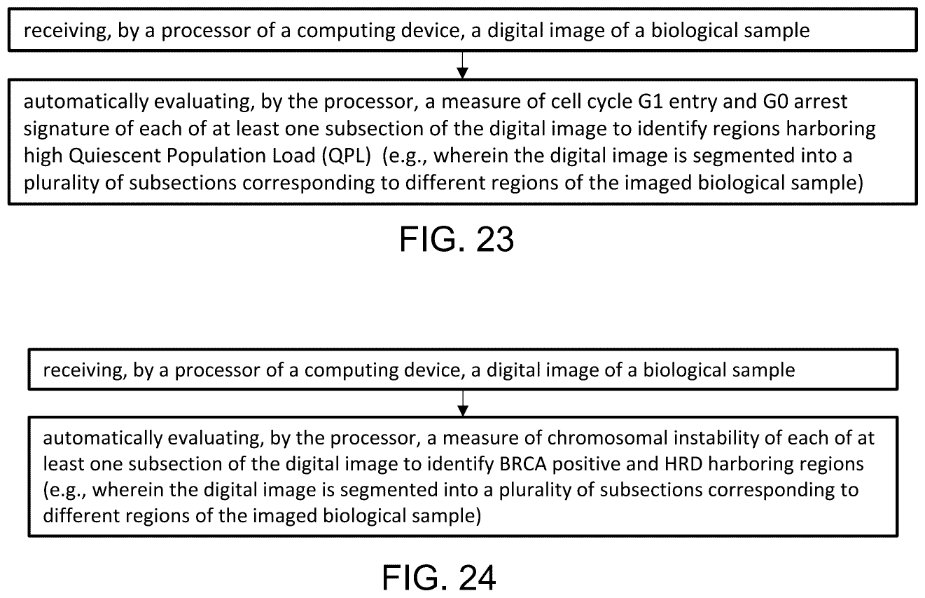

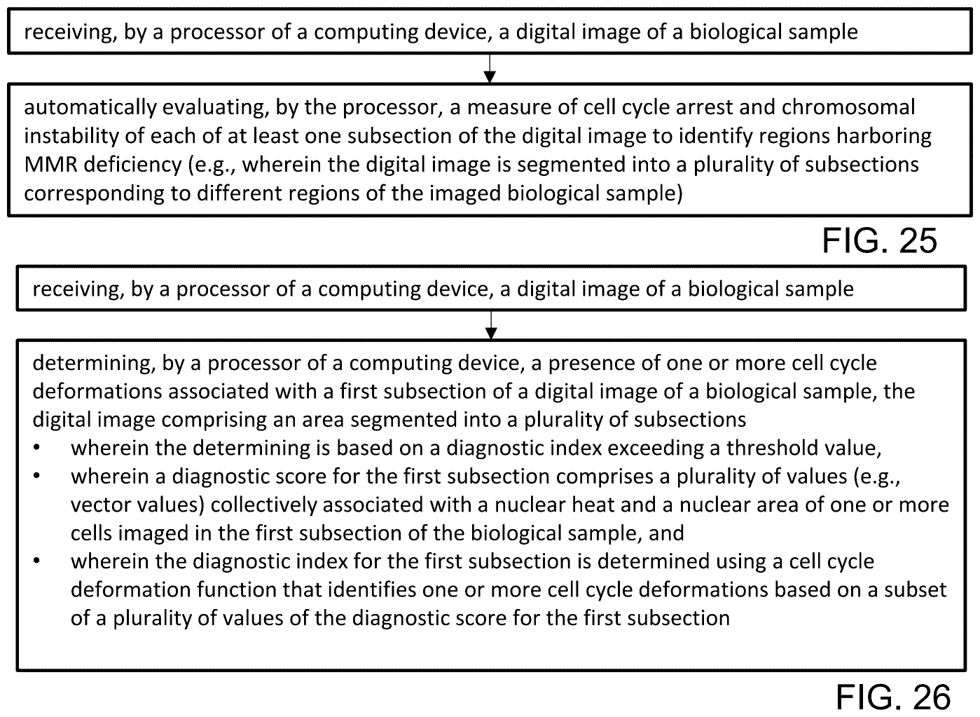

In certain embodiments, one or more of the other devices described in U.S. Pat. No. 10,535,434 may be used in the methods and systems described herein—for example, the exemplary devices for detecting molecular imprints of cancerous cells from skin lesions (FIGS. 20-22 of U.S. Pat. No. 10,535,434), the exemplary full body scanner for detecting cancer (FIG. 23 of U.S. Pat. No. 10,535,434), the exemplary device for cancer detection from internal sites of a subject (e.g., not from skin) (FIG. 24 of U.S. Pat. No. 10,535,434), the exemplary device for breast cancer detection, with biopsy needle (FIG. 25 of U.S. Pat. No. 10,535,434), the exemplary device for detecting a cancer boundary during a surgical operation (FIG. 26 of U.S. Pat. No. 10,535,434), and the exemplary endoscopy, colonoscopy, colposcopy devices described in U.S. Pat. No. 10,535,434.

In certain embodiments, the methods and systems described herein may be used in real-time or near-real-time microscopy, e.g., for in vivo or ex vivo (e.g. biopsy) analysis during a surgical procedure. Components may include, for example, real-time video monitoring devices, scanners, sensors, virtual reality (VR) or augmented reality (AR) headsets or other displays, and the like, for rendering or displaying the overlays produced as described herein in real-time or near real-time.

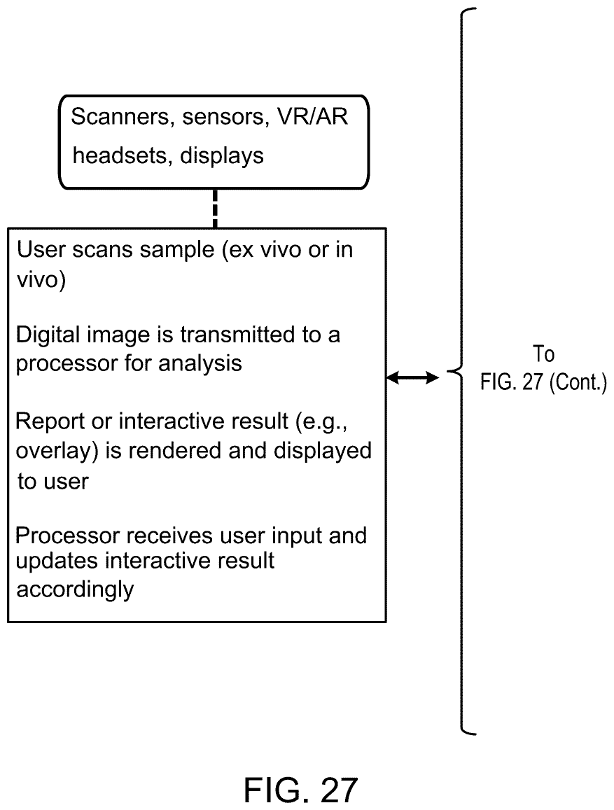

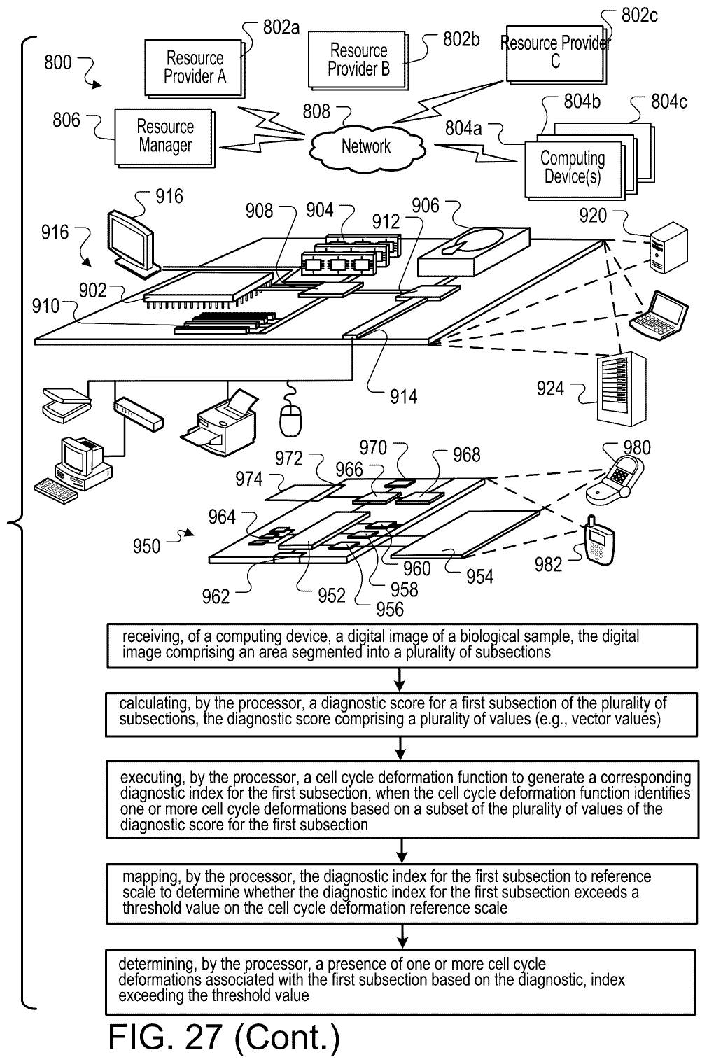

For example, FIG. 27 is a schematic diagram of a system 2700 and associated method for obtaining and analyzing a digital image of a biological sample, according to an illustrative embodiment. As shown at item 2710, the system comprises one or more scanners, sensors, VR or AR headsets, other displays, and associated equipment for obtaining digital images of a biological sample and displaying said images to one or more users. The associated equipment may include, for example, endoscopes, syringes, sponges, forceps, scalpels, microscope slides, and the like. The system also comprises a processor, network, and associated components 2730 as described in FIG. 30 and FIG. 31 of U.S. Pat. No. 10,535,434.

Element 2720 of FIG. 27 shows illustrative steps of a method involving the features of the system 2700 of FIG. 27. In an exemplary method, a user scans a sample, e.g., obtained ex vivo, for example, by biopsy as part of a surgical or non-surgical procedure. In certain other embodiments, the sample is imaged (e.g., scanned) in vivo (e.g., without separation of tissue or other biological sample from the body). In certain embodiments, obtaining or accessing the biological sample is performed for real-time image analysis or near real-time image analysis, for example, during a surgical procedure to identify the boundary of cancer or for another medical purpose. In other embodiments, obtaining or accessing the biological sample is performed for non-real time analysis.

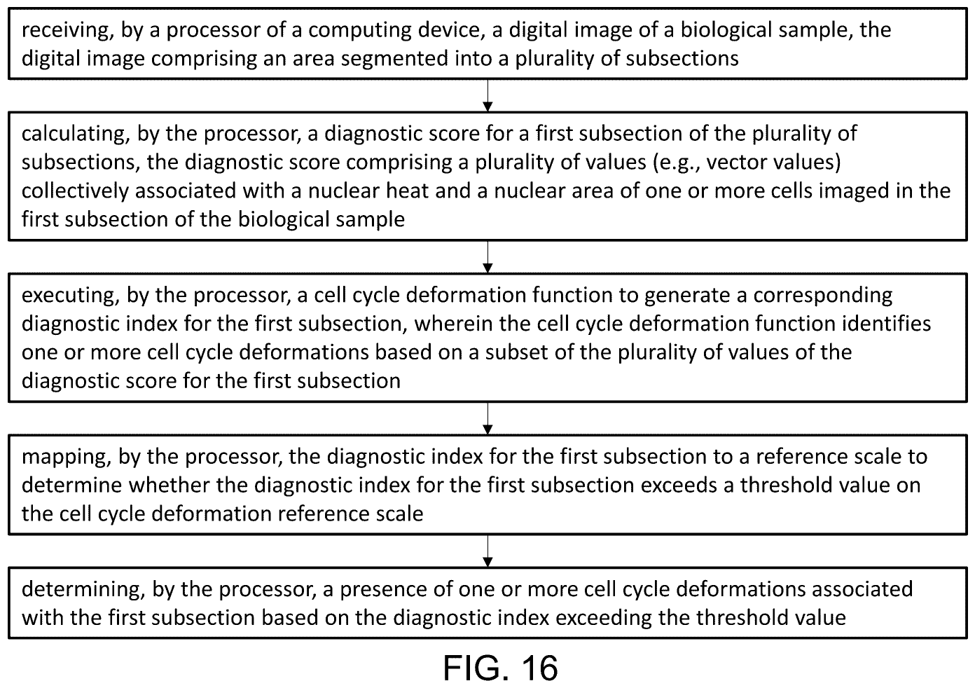

The digital image may be a still image, it may be part of a sequence or series of images, or it may be part of a video stream (e.g., live video stream) of images of the biological sample. The digital image is transmitted to a processor of a computing device for analysis. For example, the processor, network, and associated components 2730 as described in FIG. 30 and FIG. 31 of U.S. Pat. No. 10,535,434 can be used to perform steps of any of the digital image analysis methods described herein, for example, the method 2740 of FIG. 16. The processor renders a report or interactive result (e.g., overlay) which is displayed to the user via the equipment 2710. The processor may receive user input and may update the interactive result accordingly—for example, by receiving and denoting annotations on the overlay, by highlighting areas of interest identified by the user via a user interface device (e.g., by clicking a mouse, touching a touch pad display, hovering a cursor over a certain location, and the like). Graphical user interface widgets such as text boxes, sliders, drop down menus, buttons, icons, menus, radio buttons, scrollbars, windows, and the like, may be presented to the user for selection and interaction via the display.

While apparatus, systems, and methods have been particularly shown and described with reference to specific preferred embodiments, it should be understood by those skilled in the art that various changes in form and detail may be made therein without departing from the spirit and scope of the subject matter recited in the appended claims.

FIG. 30 is a flow diagram of an example process for automatically evaluating a measure of cell cycle G1/S deregulation and cell cycle G1 phase entry deregulation for each of at least one subsection of a digital image. For convenience, the process will be described as being performed by a system of one or more computers located in one or more locations. First, the system receives, by a processor, a digital image of a biological sample. Next, the system automatically evaluates a measure of cell cycle G1/S deregulation and cell cycle G1 phase entry deregulation of at least one subsection of the digital image to identify regions comprising one or more of BRAF and NRAS mutations.

In some implementations, the system identifies regions comprising BRAF gene mutations only.

In some implementations, the system identifies regions comprising NRAS gene mutations only.

In some implementations, the system identifies regions comprising both BRAF and NRAS gene mutations.

The system can be applied in any of a variety of possible applications. For instance, the system can be applied to perform BRAF/NRAS mutational status prediction from digitized whole slide images of H&E stained malignant melanomas.

For context, globally, there were over 300,000 new melanoma cases in 2020, resulting in circa 57,000 deaths. In the United States, it is the fifth most common cancer, with an overall 5-year survival rate of 93.7% and a much less favourable survival rate of 31.9% for those with distant disease. The most frequent genetic aberrations in cutaneous melanomas are BRAF and NRAS mutations, which offer both therapeutic and prognostic guidance. Testing typically revolves around sequencing or immunohistochemistry, which can be time-consuming, tissue destructive, costly and not universally accessible. The system described in this specification circumvents these issues.

To illustrate the efficacy of the system described in this specification, a study was designed to use the system on unannotated whole slide images (WSIs) of H&E-stained malignant melanoma. Unlike conventional artificial intelligence-based approaches, the system uses underlying prediction criteria that are based on deterministic, hard-coded observational relationships of continuous scales (digital biomarkers). The system maps these features to tumor-related perturbations in biological pathways and translated them into a numerical scale which enables the identification of BRAF and NRAS mutational status. 71 independent cases (totaling 139 WSIs; 1-3 slides/case; median=2) of cutaneous malignant melanoma from biopsy, resection and lymph node specimens were analyzed. Wild-type, BRAF and NRAS mutant cases were approximately equally represented (n=22, 19 and 24, respectively).

The system showed excellent performance in predicting wild type vs. BRAF/NRAS mutant cases: Cohen's kappa=0.90 (95% CI 0.78-1.01), p<0.0001, 95% accuracy, 95% sensitivity, 95% specificity, 98% PPV and 91% NPV. An 8% patient case level attrition rate (cases with no analyzable slides) was observed.

The system was able to classify malignant melanoma specimens into wild type vs. BRAF/NRAS mutant cases with a high degree of accuracy. The system can streamline current clinical testing workflows by delivering faster diagnostic turnaround times at potentially reduced cost, whilst sparing tissue.

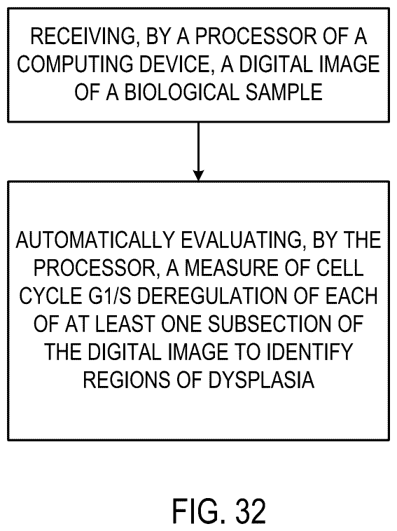

FIG. 32 is a flow diagram of an example process for automatically evaluating a measure of cell cycle G1/S deregulation for each of at least one subsection of a digital image. For convenience, the process will be described as being performed by a system of one or more computers in one or more locations. First, the system receives, by a processor, a digital image of a biological sample. Next, the system automatically evaluates a measure of cell cycle G1/S deregulation of each of at least one subsection of the digital image to identify regions of dysplasia.

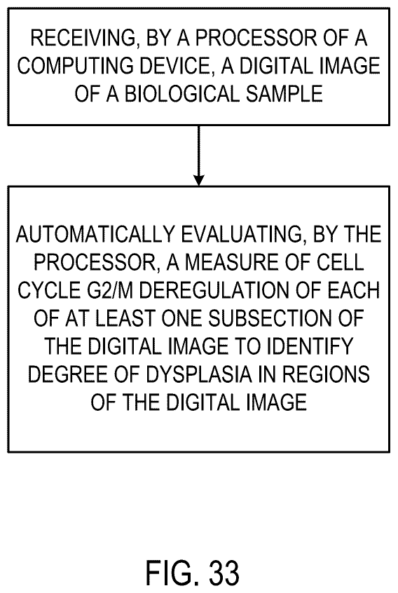

FIG. 33 is a flow diagram of an example process for automatically evaluating a measure of cell cycle G2/M deregulation of each of at least one subsection of a digital image. For convenience, the process will be described as being performed by a system of one or more computers in one or more locations. First, the system receives, by a processor, a digital image of a biological sample. Next, the system automatically evaluates a measure of cell cycle G2/M deregulation of each of at least one subsection of the digital image to identify degree of dysplasia in regions of the digital image.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A computer-implemented method comprising:
receiving, by a processor of a computing device, a digital image of a biological sample, the digital image comprising an area segmented into a plurality of subsections;
calculating, by the processor, a diagnostic score for a first subsection of the plurality of subsections, the diagnostic score comprising a plurality of values collectively associated with a nuclear heat and a nuclear area of one or more cells imaged in the first subsection of the biological sample;
executing, by the processor, a cell cycle deformation function to generate a corresponding diagnostic index for the first subsection, wherein the cell cycle deformation function identifies one or more cell cycle deformations based on a subset of the plurality of values of the diagnostic score for the first subsection;
mapping, by the processor, the diagnostic index for the first subsection to a reference scale to determine whether the diagnostic index for the first subsection exceeds a threshold value on the cell cycle deformation reference scale; and
determining, by the processor, a presence of one or more cell cycle deformations associated with the first subsection based on the diagnostic index exceeding the threshold value.

2. (canceled)

3. (canceled)

4. (canceled)

5. (canceled)

6. (canceled)

7. (canceled)

8. (canceled)

9. (canceled)

10. (canceled)

11. The computer-implemented method of claim 1, wherein the cell cycle deformation function calculates cell cycle S-phase deregulation to identify regions of high grade cancer.

12. The computer-implemented method of claim 1, wherein the cell cycle deformation function calculates active cell cycle to identify regions of high percentage of Ki67 expression.

13. The computer-implemented method of claim 1, wherein the cell cycle deformation function calculates skew in nuclear size and chromosomal instability to identify regions of high DNA Ploidy.

14. The computer-implemented method of claim 1, wherein the cell cycle deformation function calculates structural distortion in stroma and cell cycle arrest to identify regions of high stromal TILs.

15. The computer-implemented method of claim 1, wherein the cell cycle deformation function calculates cell cycle G1/S deregulation and arrest signature to identify HER2 positive/amplified regions.

16. The computer-implemented method of claim 1, wherein the cell cycle deformation function calculates cell cycle G1/S deregulation and arrest signature to identify HR positive/overexpressed regions.

17. The computer-implemented method of claim 1, wherein the cell cycle deformation function calculates cell cycle G1 entry and G0 arrest signature to identify regions harboring high Quiescent Population Load (QPL).

18. The computer-implemented method of claim 1, wherein the cell cycle deformation function calculates chromosomal instability to identify BRCA positive and HRD harboring regions.

19. The computer-implemented method of claim 1, wherein the cell cycle deformation function calculates cell cycle arrest and chromosomal instability to identify regions harboring MMR deficiency.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method of claim 31, wherein the automatically evaluating comprises computing percentage of tissue area with high skew in (C_L)max for each of the at least one subsection.

33. (canceled)

34. The method of claim 33, wherein the automatically evaluating comprises computing shape (<C>) deviation for each of the at least one subsection.

35. A method comprising:
receiving, by a processor of a computing device, a digital image of a biological sample, and
automatically evaluating, by the processor, a measure of cell cycle G1/S deregulation and arrest signature of each of at least one subsection of the digital image to identify HER2 positive/amplified regions.

36. The method of claim 35, wherein the automatically evaluating comprises computing shape (<C_L>) divergence for each of the at least one subsection.

37. (canceled)

38. (canceled)

39. (canceled)

40. A method comprising:
receiving, by a processor of a computing device, a digital image of a biological sample, and
automatically evaluating, by the processor, a measure of cell cycle G1 entry and G0 arrest signature of each of at least one subsection of the digital image to identify regions harboring high Quiescent Population Load (QPL).

41. The method of claim 39, wherein the automatically evaluating comprises computing (<C>max−<C>min)×(skew in <C>max) for each of the at least one subsection.

42. (canceled)

43. (canceled)

44. (canceled)

45. (canceled)

46. (canceled)

47. (canceled)

48. (canceled)

49. (canceled)

50. The computer-implemented method of claim 1, wherein the cell cycle deformation function calculates cell cycle G1/S deregulation and cell cycle G1 phase entry deregulation to identify regions comprising one or more of BRAF and NRAS gene mutations.

51. (canceled)

52. The method of claim 50, wherein the automatically evaluating comprises computing shape (<C>max, <C>min, <C>) divergence for each of the at least one subsection.

53. The computer-implemented method of claim 1, wherein the cell cycle deformation function calculates cell cycle G1/S deregulation to identify regions of dysplasia.

54. (canceled)

55. (canceled)

56. The computer-implemented method of claim 1, wherein the cell cycle deformation function calculates cell cycle G2/M deregulation to identify degree of dysplasia in regions.

57. (canceled)

58. (canceled)

59. (canceled)

60. (canceled)
